(19) United States
(12) Patent Application Publication
(10) Pub. No.: US 2011/0182973 A1
(43) Pub. Date: Jul. 28, 2011

(54) POLYMERASE INHIBITORS AND THE USE THEREOF FOR THE TREATMENT OF TUMORS

(75) Inventors: Monika Schaefer-Korting, Berlin (DE); Hans-Dieter Hoeltje, Berlin (DE)

(73) Assignees: FREIE UNIVERSITAET BERLIN, Berlin (DE); HEINRICH-HEINE-UNIVERSITAET DUESSELDORF, Duesseldorf (DE)

(21) Appl. No.: 12/599,614

(22) PCT Filed: May 13, 2008
(86) PCT No.: PCT/DE2008/000818

§ 371 Date: Jul. 7, 2010

(30) Foreign Application Priority Data
May 11, 2007 (EP) 07090098.0
May 30, 2007 (EP) 07090106.1
Jul. 30, 2007 (EP) 07075656.4
Aug. 31, 2007 (DE) 10 2007 041 968.8
Sep. 3, 2007 (DE) 07075752.1
Sep. 13, 2007 (DE) 10 2007 044 849.1
Sep. 28, 2007 (EP) 07075853.7
Jan. 4, 2008 (DE) 10 2008 003 477.0

(51) Int. Cl. A61K 9/127; C07D 473/16; C07F 9/6561; C07D 405/04; A61K 31/52; A61K 31/675; A61K 31/513; A61P 35/00; B82Y 5/00

(52) U.S. Cl. 424/450; 544/277; 544/244; 544/312; 544/314; 544/276; 514/263.23; 514/80; 514/274; 977/773; 977/797; 977/906

(57) ABSTRACT

The invention relates to polymerase inhibitors, particularly polymerase alpha inhibitors, and the use thereof in the treatment of cell growth disorders, particularly tumor disorders, preferably actinic keratoses, basal cell carcinomas, and/or spinocellular carcinomas.

The invention relates to polymerase inhibitors, particularly polymerase alpha inhibitors, and the use thereof in the treatment of cell growth disorders, particularly tumor diseases, preferably actinic keratoses, basal cell carcinomas, and/or spinocellular carcinomas.

The term tumor disease is being used synonymously with cancer. More specifically, such diseases are understood to be malignant tumors. In a stricter sense, malignant hemoblastoses are also defined as cancer among experts (e.g. leukemia as blood cancer). Cancer is being used as a collective term for a variety of related diseases where body cells can undergo uncontrolled growth and division and displace or flank healthy tissue or infiltrate or destroy the latter.

A wide variety of agents and methods for the treatment of tumor diseases are known in the prior art. Tumor diseases are among the most frequent causes of death, and therefore it must be assumed that a large number of these agents and methods are being employed with only limited success. Moreover, the above-mentioned agents and methods are known to have quite a number of side effects. The prior art has disclosed anticancer agents where the side effects unfortunately are of considerable importance to the patient, thereby substantially reducing the quality of life.

Malignant skin tumors, also referred to as malignant tumors or skin cancer or malignant melanoma, are among the most frequent tumor diseases in humans. They frequently develop on skin areas exposed to solar radiation, e.g. on the head, upper body, on the calves of cyclists. In general, people spending a lot of time in the open air, such as farmers, seamen or fishermen, are frequently affected. As specific sections of the population equate tanned skin with healthy relaxation and dynamic lifestyle, there has been a significant increase of cancerous diseases over the years. Especially in countries with high solar radiation, such as Australia, there has been an alarming increase in the frequency of skin cancer diseases. But also in Germany it is about 8,000 people who develop an especially malignant form of skin cancer, the malignant melanoma (black skin cancer). Still more important is UV light in the development of non-melanoma skin cancer (actinic keratoma, spinocellular carcinoma and basal cell carcinoma). The method of choice in basal cell carcinoma and spinocellular carcinoma is surgical removal of the tumor. The malignant melanoma is among those tumors with the most rapid metastasation. For this reason, therapy of such a tumor also necessitates surgical removal of metastases. Following surgery, a so-called immunotherapy is frequently performed. The intention is to strengthen the endogenous defense particularly with the aid of interferon. Today, chemotherapy using cytostatic agents is employed as a single therapy to only a very limited extent. The reason for this is that, among other things, such agents do not allow a locally confined treatment of the tumors specified above.

Furthermore, polymerase inhibitors are known in the prior art, which can inhibit the reduplication of DNA or RNA and, for example, counteract the growth of viruses or degenerate cells. Although the effect of these so-called nucleoside analogs has been extensively discussed in the literature, the practical success is meager and constantly remains below the expectations. Even though the use of such structures in virus or cancer therapy appeared obvious, a person skilled in the art, due to the contradictory state of the art and the numerous negative results during practical use of these structures, has had no motivation to employ nucleosides/nucleoside analogs in cancer therapy. In particular, it has been found that even minor modifications in the structure of the nucleosides give rise to a substantial loss of activity or a dramatic increase of side effects. Such problems cannot be overcome by means of routine tests with the intention of finding new structures because the number of possible structures to be used is virtually infinite.

The object of the invention was therefore to provide nucleosides, in particular specific selected nucleosides, which have a surprisingly good effect on selected tumors.

Quite surprisingly, the object of the invention can be accomplished by providing an antitumor agent, especially for the treatment of dermal tumors, selected from the group comprising compounds according to the general formulas (1) to (16):

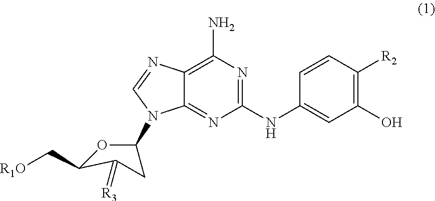

R₁═H or mono- or di- or triphosphate
R₂=butyl or pentyl or hexyl or isohexyl derivative
R₃═O or NOH

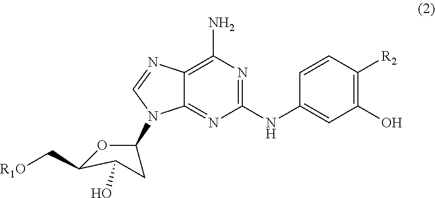

R₁═H or mono- or di- or triphosphate
R₂=butyl or pentyl or hexyl or isohexyl derivative

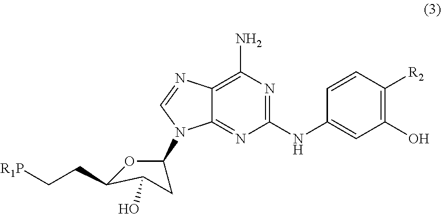

P=phosphonate
R₁═H or mono- or diphosphate
R₂=butyl or pentyl or hexyl and/or isohexyl derivative

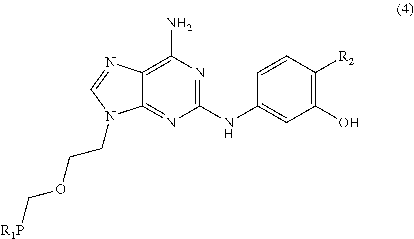

P=phosphonate
R₁═H or mono- or diphosphate
R₂=butyl or pentyl or hexyl and/or isohexyl derivative

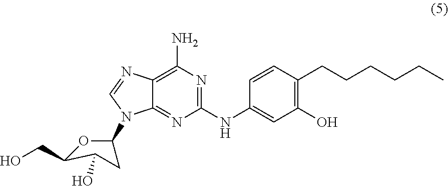

2-(4-Hexyl-3-hydroxyphenylamino)-2′-deoxyadenosine

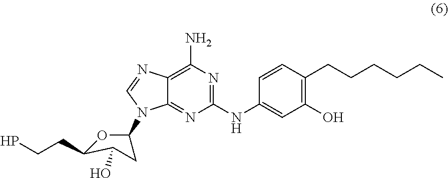

P=phosphonate
2-(4-Hexyl-3-hydroxyphenylamino)-9-[4-hydroxy-5-(2-phosphonoethyl)tetrahydro-2-furyl]adenine

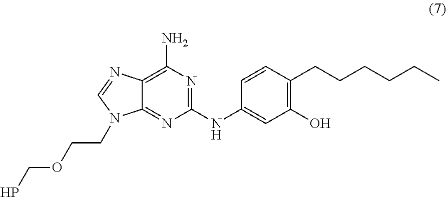

P=phosphonate
2-(4-Hexyl-3-hydroxyphenylamino)-9-[2-(phosphonomethoxy)ethyl]adenine

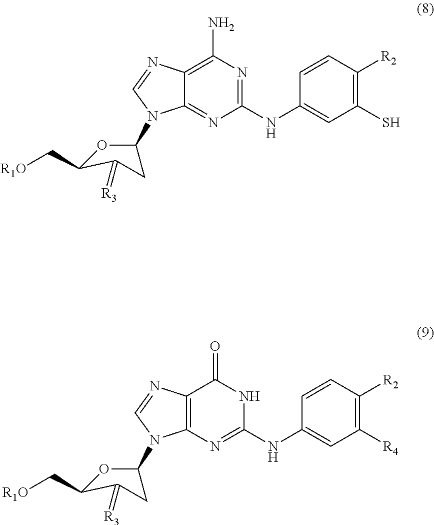

R₁═H or mono- or di- or triphosphate
R₂=butyl or pentyl or hexyl or isohexyl derivative
R₃═O or NOH
R₄═OH or SH

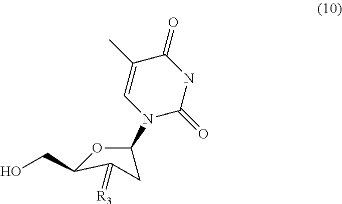

R₁═O or NOH
3′-deoxy-3′-oxothymidine or
3′-deoxy-3′-hydroxyliminothymidine

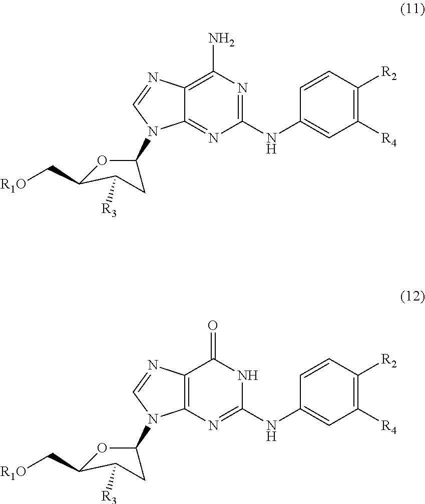

R₁═H or mono- or di- or triphosphate
R₂=butyl or pentyl or hexyl or isohexyl derivative
R₃═OH or H
R₄═OH or SH

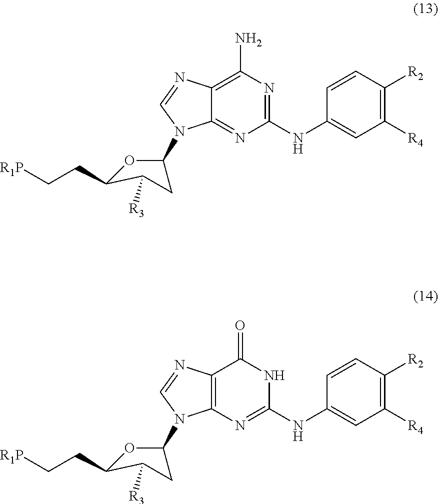

P=phosphonate
R₁═H or mono- or diphosphate
R₂=butyl or pentyl or hexyl or isohexyl derivative
R₃═OH or H
R₄═OH or SH

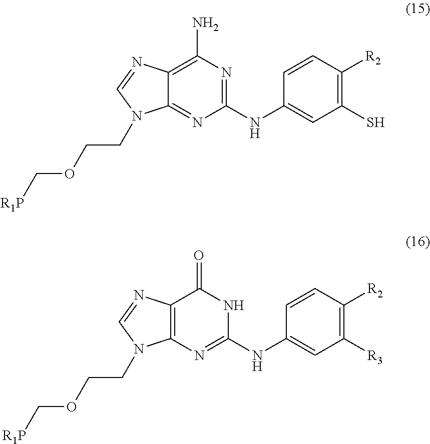

P=phosphonate
R₁═H or mono- or diphosphate
R₂=butyl or pentyl or hexyl or isohexyl derivative
R₃═OH or SH

The teaching according to the invention combines an integrated group of inventions interconnected by a single general inventive idea. The claimed compounds can be understood as alternatives having common properties or effects. All alternatives belong to a well-known class of chemical compounds in the field of the invention.

Moreover, the teaching according to the invention represents a selection invention wherein particular nucleosides with surprising properties regarding the effect on tumors, especially skin tumors, or skin cell growth disorders are selected.

In another preferred aspect of the invention the above object is accomplished by providing an agent specifically for the treatment of cell modifications, preferably in a skin area, in particular selected from the group comprising actinic keratosis, malignant melanomas, spinocellular carcinomas and/or basal cell carcinomas, which agent has improved antiproliferative activity compared to diclophenac and/or 5-FU and is selected from the group comprising compounds according to the general formulas 1 to 4:

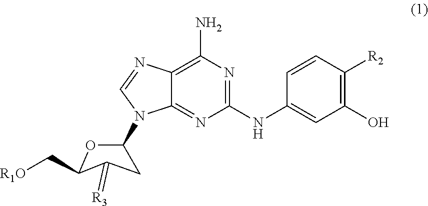

R₁═H or mono- or di- or triphosphate
R₂=butyl or pentyl or hexyl or isohexyl derivative
R₃═O or NOH

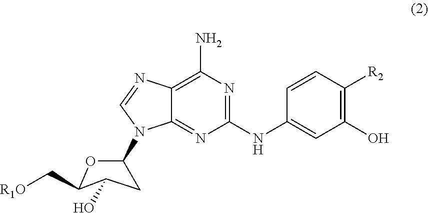

R₁═H or mono- or di- or triphosphate
R₂=butyl or pentyl or hexyl or isohexyl derivative

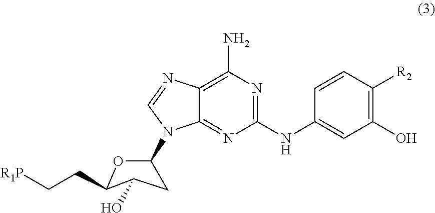

P=phosphonate
R₁═H or mono- or diphosphate
R₂=butyl or pentyl or hexyl and/or isohexyl derivative

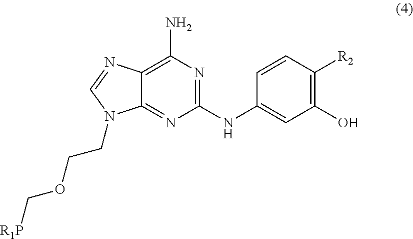

P=phosphonate
R₁═H or mono- or diphosphate
R₂=butyl or pentyl or hexyl and/or isohexyl derivative

Quite surprisingly, the above-mentioned compounds according to the general formulas 1 to 4 are highly suitable for use in the treatment of cell modifications, preferably in an area of the skin. The agents of the invention modulate, especially inhibit, a polymerase, particularly polymerase alpha. By virtue of the above technical teaching, a person of average skill in the art will be able to treat new patient collectives and new diseases which could not be treated with previous agents of the prior art.

A skin area in the meaning of the invention is any area which is associated with the epidermis, dermis, subcutis or cutaneous appendages, ridged skin or meshed skin, completely or partially forms them or is present as such or associated with them. In the meaning of the invention this can be tissue in the breast, colon and rectum or in the urinary bladder. In the meaning of the invention, however, skin in preferred embodiments is not specifically or exclusively understood as the external organ of a human or animal organism, which serves as a boundary of inside and outside. In the meaning of the invention a skin area in preferred embodiments includes components of the epidermis, dermis or subcutis. According to the invention the epidermis is constituted of the following layers: horny layer (stratum corneum), clear layer (stratum lucidum), granular layer (stratum granulosum), prickle cell layer (stratum spinosum) and/or basal layer (stratum basale). Any modification of cells in this area is a cell modification in a skin area in the meaning of the invention.

The dermis (corium), which is also a component of the skin areas according to the invention, is predominantly constituted of connective tissue fibers and serves in nourishing and anchoring the epidermis. The capillarized vascular system in the boundary zone to the epidermis as well as the sebaceous and perspiratory glands are included in the skin area in the meaning of the invention. The dermis in the meaning of the invention can be divided into a stratum papillare and a stratum reticulare.

Furthermore, a skin area in the meaning of the invention can be any area, i.e. any location, in or on the subcutis or of a tissue inside the body or of an organ or component of an organ. A tissue barrier which forms a boundary between an organ and the surrounding structures can be a skin in the meaning of the invention.

Furthermore, the term skin area according to the invention comprises cutaneous appendages such as hair, sebaceous glands, hair follicle muscles, nails, horny material and perspiratory glands, especially the exocrine and apocrine perspiratory glands, as well as the lactiferous glands. Any cell modification, especially cell growth deviating from normal, can be treated with the agents according to the invention, and in a preferred fashion this is not restricted to external skin areas.

However, skin areas in the meaning of the invention may also concern the ridged skin, such as occurring on fingers or the sole, or the meshed skin and cutaneous appendages associated therewith.

Complete or partial swellings or tumors may form on all the types of skin mentioned above. That is, an increase in tissue volume may arise due to any cause. A reduction in tissue volume is of course also possible. In one embodiment of the invention, such changes in tissue volume are understood to be cell modifications in the meaning of the invention. Also, changes in the activity of cells, with no increase or reduction of the tissue volume taking place, can be cell modifications in the meaning of the invention, e.g. in those cases where particular modified cells infiltrate or penetrate some other cell tissue which may or may not be part of a skin area. Similarly, these processes are understood to be cell modifications in the meaning of the invention. Moreover, an inflammation which affects a skin area or is adjacent thereto is a cell modification in the meaning of the invention.

Preferred cell modifications in a skin area are actinic keratosis, malignant melanomas, spinocellular carcinomas or basaliomas.

Actinic keratosis in the meaning of the invention is a change of the skin, especially the horny epidermis, caused in particular by light-induced lesions. Actinic keratosis may develop malignancy and result in skin cancer (facultative precancerosis). Actinic keratosis affects humans at any year of life, but in a preferred embodiment of the invention especially those in their second half of life. In a preferred embodiment of the invention, actinic keratosis particularly occurs in the face, on the back of hands, on the forehead, upper head/bald head, nose and ear. Apparently, actinic keratosis is not restricted to humans but rather affects all creatures, especially mammals, and more preferably domestic dogs and cats. It is of course also possible that actinic keratosis occurs in an organism, simultaneously or time-shifted, as a cell modification in a skin area, associated or non-associated with other dermal changes such as precancerous stages (e.g. Bowen's disease).

Basalioma in the meaning of the invention is a basal cell carcinoma or precursor of such a cancer, which can also be referred to as basal cell epithelioma. More specifically, this cell modification may have its origin in the basal epidermal cell layers. It may also damage the surrounding non-dermal tissue and infiltrate the bone cells. Any treatment of such a cell modification is understood to be treatment of a cell modification of a skin area in the meaning of the invention, especially because the cell modification has its origin in an area of the skin, namely, the epidermal cell layers.

Spinocellular carcinoma in the meaning of the invention is a cell modification in a skin area, which is triggered particularly by UV light and therefore especially appears in areas of the body, which—much like in basaliomas—are exposed to light, such as the face. In preferred embodiments of the invention, precanceroses such as actinic keratosis and Bowen's disease can be regarded as spinalioma. In the meaning of the invention the metastases formed by a spinalioma or e.g. by a basalioma are also regarded as cell modification in a skin area.

A malignant melanoma in the meaning of the invention is any degeneration, especially malignant degeneration, of pigment cells (melanocytes). Malignant melanomas show a strong tendency of spreading metastases via the lymphatic and blood circulation systems at an early stage. As for cell modifications in a skin area the invention therefore also comprises the treatment of metastases arising from a malignant melanoma.

The agent according to the invention can suitably be used to influence, especially inhibit, particularly the polymerase of cells modified as a result of enhanced growth or a change in their biochemical activity. The agents according to the invention can therefore be used in the context with influencing, reduplicating or repairing DNA or RNA, especially in tumors. In a preferred fashion the effect on tumors is not restricted to skin tumors. Surprisingly, it was found that the agents according to the invention can be used particularly in local therapies of tumors occurring in an area of the skin, and these tumors can be primary or secondary tumors in said skin area. Accordingly, preferred targets of the agents according to the invention are the polymerases present in tumor tissue, i.e. especially in the cells. Within the tumor tissue or adjoining tissue, cells can be concerned that are present in a non-modified or not yet modified, e.g. degenerate, form. Although there are agents already available which can be used for similar tumors, such as diclofenac and/or 5-FU (whose effectiveness against tumor cells is used in their classification in the prior art), it was very surprising to find that the structures according to the invention can be used in the treatment of in particular skin tumors in an especially effective manner. The agents according to the invention preferably have high affinity to polymerase alpha which thereby no longer binds to a primase so that replication of the main and successive strands cannot take place. It will be appreciated that the agents according to the invention can be employed against all polymerases, especially eukaryotic polymerases, preferably classes A, B, X and Y. What is particularly surprising, however, is the effect of the agents on polymerase alpha in the context with treatment of the above-mentioned cell modifications. In preferred embodiments of the invention the agents according to the invention therefore can also be referred to as DNA polymerase inhibitors.

Apart from the use mentioned above, the structures according to the invention can also be used as lead structures for the development of other polymerase inhibitors, especially polymerase alpha inhibitors. It will be appreciated that the invention is not only restricted to the agents according to the invention but also relates to functionally analogous molecules which, compared to the molecules preferably in accordance with formulas 1 to 4 or preferably in accordance with formulas 1 to 7, exhibit a similar behavior in solving the problem according to the invention. More specifically, functional analogs in the meaning of the invention can be regarded as equivalents which can be generated in different ways but essentially achieve the same function on essentially the same way and produce essentially the same result as preferably the compounds in accordance with general formulas 1 to 4 as well as 5, 6 or 7. The term “functional analogs” in the context with the teaching of the invention is therefore sufficiently clear because protection of an agent defined via the result to be achieved is ruled out. In particular, the question whether functional analogs, i.e. equivalents, are encompassed by the teaching of the invention is answered according to whether the functional analogs would solve the problem forming the basis of the invention, though using modified but objectively equally effective agents, and whether a person skilled in the art, by virtue of expertise, would have the capability of finding modified agents (functional analogs or variants) having the same effect. The reflections to be made by an artisan to this end would have to be based on the meaning of the teaching protected in the claims so that the artisan would have considered the use of the functional analogs and variants as a solution of the object of the invention which is equivalent to the solution of said object with the agents preferably in accordance with general formulas 1 to 4. Accordingly, functionally analogous molecules are those which would be found by a person skilled in the art as having the same effect, to which end a person skilled in the art would have to make reflections particularly based on the meaning of the teaching protected in the claims, so that a person skilled in the art would consider the functional analogs or variants as a solution which is equivalent to the solution according to the molecules claimed according to the invention, preferably in accordance with general formulas 1 to 4.

The terms “functional analogs” or “variants” and “essentially the same function”, “in essentially the same way” and “essentially the same result” are not relative terms because these expressions have a generally recognized meaning in the art of biology. The terms “functional analogs” and “variants” are understood as equivalents according to the invention and have been repeatedly defined in the jurisdiction (inter alia, in the draft of the International Patent Law Harmonization Treaty), so that these terms are sufficiently clear. These terms therefore have a generally recognized meaning, so that replacement with more precise information is not required. The term “essentially” has been approved according to the definition in the International Patent Law Harmonization Treaty because it serves as a so-called “softening means” of claims with exceedingly strict or narrow definition. The intention of using the term “essentially” is to avoid that a molecule which is a variant of the molecules claimed according to the invention and furnishes essentially the same function in essentially the same way with essentially the same result is no longer encompassed by the claims if, for example, it fails to furnish the exactly identical result or if slightly modified conditions for the use of such functional analogs or variants are required for optimum use. In the meaning of the invention the term “in essentially the same way” therefore implies that the functional analogs or variants can be used particularly for the treatment of actinic keratoses, malignant melanomas, spinocellular carcinomas and/or basal cell carcinomas on areas of the skin.

In a preferred embodiment of the invention the agent according to the invention has the following structure:

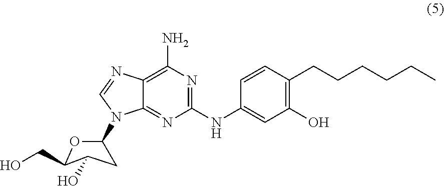

2-(4-Hexyl-3-hydroxyphenylamino)-2′-deoxyadenosine

In another preferred embodiment the agent according to the invention has the following structure:

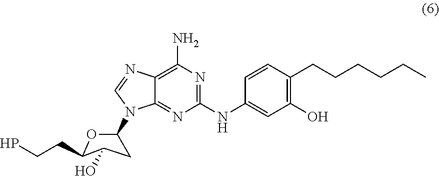

P=phosphonate
2-(4-Hexyl-3-hydroxyphenylamino)-9-[4-hydroxy-5-(2-phosphonoethyl)tetrahydro-2-furyl]adenine

In another preferred embodiment of the invention the agent according to the invention has the following structure:

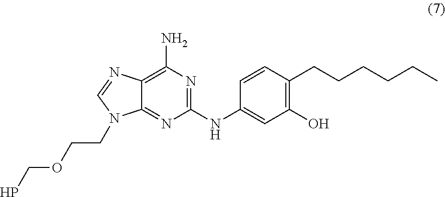

P=phosphonate
2-(4-Hexyl-3-hydroxyphenylamino)-9-[2-(phosphonomethoxy)ethyl]adenine

The invention also relates to agents that are functionally analogous to the compounds in accordance with general formulas 1 to 7 and selected from the group comprising

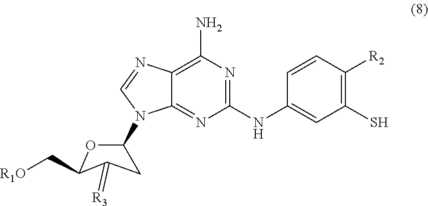

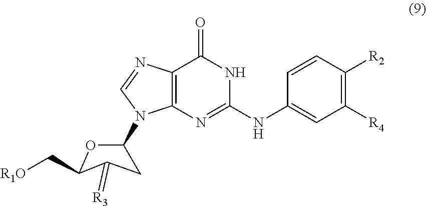

R₁═H or mono- or di- or triphosphate
R₂=butyl or pentyl or hexyl or isohexyl derivative
R₃═O or NOH
R₄═OH or SH

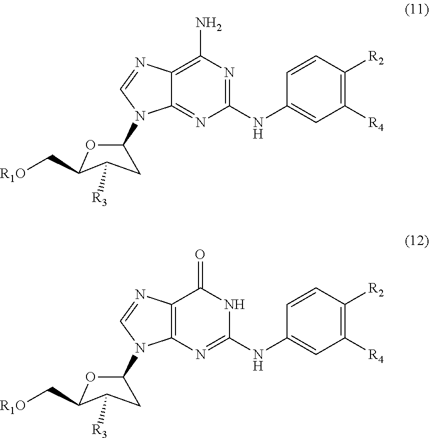

R₁═H or mono- or di- or triphosphate
R₂=butyl or pentyl or hexyl or isohexyl derivative
R₃═OH or H
R₄═OH or SH

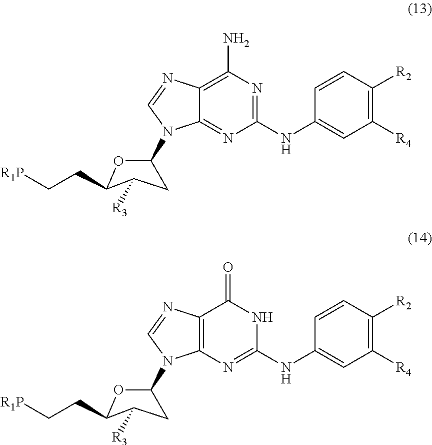

P=phosphonate
R₁═H or mono- or diphosphate
R₂=butyl or pentyl or hexyl or isohexyl derivative
R₃═OH or H
R₄═OH or SH

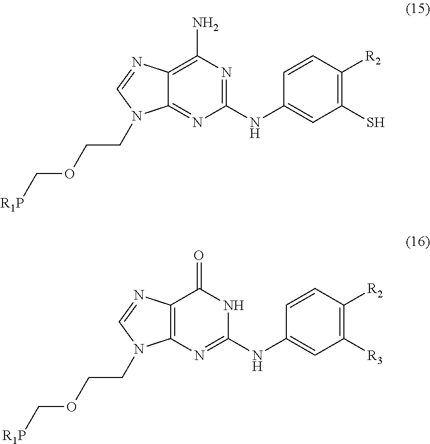

P=phosphonate
R₁═H or mono- or diphosphate
R₂=butyl or pentyl or hexyl or isohexyl derivative
R₃═OH or SH

The invention also relates to a compound of general formula 10

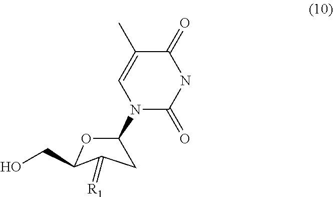

R₁═O or NOH
3′-Deoxy-3′-oxothymidine or 3′-deoxy-3′-hydroxyliminothymidine

with HM-1 being claimed in a first medical indication and the HM-1 oxime derivative in a use for the treatment of tumors (second medical indication). Accordingly, both HM-1 and HM-1 oxime derivative are claimed as antitumor agents, especially for cell growth disorders of the inner and outer skin. It was completely surprising to find that the compound in accordance with general formula 10 can be used in the prophylaxis or therapy or aftercare of tumor diseases, especially skin tumors. The compounds in accordance with general formulas 1 to 16 represent a unit because they solve the basic problem of the invention. The compounds are interconnected in such a way that a single general inventive idea is accomplished. There is a technical relationship between the compounds in accordance with general formulas 1 to 16 such that all of these compounds can be used as antitumor agents, especially for the treatment of skin tumors. The agents according to the invention can be applied in such a way that growth, metastasation, invasion, infiltration and/or angiogenesis of a tumor is prevented or inhibited. The effect of the inventive compounds on tumors with respect to their prophylactic or therapeutic potential can be seen e.g. in an inhibition of tumor growth or in a reduction of angiogenesis or metastasation or otherwise. The administered dose will depend on the age, health and weight of the recipient, degree of the disease, type of required simultaneous treatment, frequency of the treatment and type of the desired effects, and side-effects. The respective dose or dosage range for administering the pharmaceutical agent according to the invention is sufficiently high in order to achieve the desired prophylactic or therapeutic antitumoral effect. In this context, the dose should not be selected in such a way that undesirable side effects would dominate. In general, the dose will vary with the patient's age, constitution, sex and, of course, depending on the severity of the disease. The individual dose can be adjusted both with respect to the primary disease and with respect to the occurrence of additional complications. Using well-known means and methods, the exact dose can be determined by a person skilled in the art, e.g. by determining the tumor size, number of leukocytes or the like as a function of dosage or as a function of the treatment regime or pharmaceutical carrier and the like. Depending on the patient, the dose can be selected individually. For example, a dose of pharmaceutical agent just tolerated by a patient can be such that the range thereof in plasma or locally in particular organs is from 0.1 to 100,000 μM, preferably between 1 and 1,000 μM. Alternatively, the dose can be calculated relative to the body weight of the patient. In this event, a typical dose of pharmaceutical agent would have to be adjusted e.g. in a range of more than 0.1 mg per kg body weight, preferably between 0.1 and 5,000 mg/kg. Furthermore, however, it is also possible to determine the dose on the basis of particular organs rather than the whole patient. For example, this would be the case when placing the pharmaceutical agent according to the invention e.g. in a biopolymer incorporated in the respective patient near specific organs by means of surgery. A number of biopolymers capable of liberating the molecules in a desirable manner are known to those skilled in the art. For example, such a gel may include 1 to 1,000 mg of the inventive compounds or pharmaceutical agent per ml of gel composition, preferably between 5 and 500 mg/ml, and more preferably between 10 and 100 mg/ml.

In this event, the therapeutic agent is administered as a solid, gel-like or liquid composition. In addition to the above-specified concentrations during use of the compounds of the invention, the compounds in another preferred embodiment can be employed in a total amount of 0.05 to 500 mg/kg body weight per 24 hours, preferably 1 to 10 mg/kg body weight. Advantageously, this is a therapeutic quantity which is used to prevent or improve the symptoms of a disorder or of a responsive, pathologically physiological condition. The administered amount is sufficient to prevent or inhibit growth, metastasation, invasion, infiltration and/or angiogenesis of a tumor. The effect of the inventive compounds on tumors with respect to their prophylactic or therapeutic potential can be seen e.g. in inhibition of growth or otherwise. For example, the therapeutic effect can be such that, as a desirable side effect, particular antitumor medicaments are improved in their effect or, by reducing the dose, the number of side effects of these medicaments will be reduced as a result of applying the compounds of the invention. Of course, the therapeutic effect also encompasses direct action on the tumor. That is, however, the effect of the compounds of the invention is not restricted to eliminating tumors, but rather comprises the entire spectrum of advantageous effects in prophylaxis and therapy. Obviously, as set forth above, the dose will depend on the age, health and weight of the recipient, degree of the disease, type of required simultaneous treatment, frequency of the treatment and type of the desired effects, and side-effects. The daily dose of 0.05 to 500 mg/kg body weight can be applied in the form of a single dose or multiple doses in order to furnish the desired results. The dose levels per day can be used in prevention and treatment of a tumor disease. It will be appreciated that the amounts of active substance combined with the carrier materials to produce a single dosage form may vary depending on the patient to be treated and on the particular type of administration. In a preferred fashion, the daily dose is distributed over 2 to 5 applications, with 1 to 2 preparations (pads, tablets etc.) including an active substance content of 0.05 to 5 mg/kg body weight being administered in each application. Of course, it is also possible to select a higher content of active substance, e.g. up to a concentration of more than 500 mg/kg. It may become necessary to deviate from the above-mentioned dosages, and this depends on the nature and body weight of the host to be treated, the type and severity of the disease, the type of formulation and application of the drug, and on the time period or interval during which the administration takes place. Thus, it may be preferred in some cases to contact the organism with less than the amounts mentioned above, while in other cases the amount of active substance specified above has to be surpassed. A person of specialized knowledge in the art can easily determine the optimum dosages required in each case and the type of application of the active substances.

Accordingly, the invention relates to the claimed agents in accordance with general formulas 1 to 9 and 11 to 16, preferably for use as drugs. In a preferred fashion the agents are used as pharmaceutical preparations comprising the agent of the invention and optionally a pharmaceutically tolerable carrier.

As a result of their functional relationship, the compounds in accordance with general formulas 1 to 16 are interconnected into an integrated inventive idea in that they exhibit an effect which they have in common. The effect they have in common is represented by their surprisingly good usefulness in tumor therapy.

Accordingly, the invention in a specific aspect relates to the surprising teaching that the compounds in accordance with general formulas (1) to (16) can be used in the treatment of dermal tumors in a highly specific and particularly efficient manner. It was completely surprising to find that the inventive compounds according to the general formulas (1) to (16) are particularly useful in tumor therapy. Similar compounds have been described in the prior art, but these compounds do not exhibit such surprising properties. Hence, inventive activity related to the new use of the compounds in accordance with general formulas (1) to (16) cannot be denied on the basis of their similarity to well-known chemical compounds, because this would imply the suggestion that a person of average skill in the art would have expected that the partially new compounds—in a new use—would be equally or similarly well suited as a means for an inventive solution of the basic technical problem as the well-known compounds. The existing differences to well-known chemical compounds have a substantial influence on the properties of the compounds claimed according to the invention and therefore are of great importance to the solution of the technical problem. In contrast to the well-known compounds, the compounds in accordance with general formulas (1) to (16) have barely any or no effect at all on normal cells, but are highly toxic when contacted with cancer cells. Also, the structures according to the invention have higher lipophilicity compared to the well-known compounds. Prior art compounds, while possibly having similar lipophilicity, are disadvantageously large so that they cannot be reasonably used to interact particularly with polymerase alpha and thus treat tumors. In particular, the presence of hydroxyl groups in the structures according to the invention has not been disclosed in the context with well-known compounds. Owing to the preferred presence of the hydroxyl groups, proteins such as polymerase alpha can form at least one hydrogen bridge that initiates a particularly stable bond. It was completely surprising to find that the inventive molecules in accordance with general formulas 1 to 16 are highly suitable for use in humans, whereas merely in vitro activity e.g. on viruses has been described for similar compounds of the prior art. Likewise, the advantageous extension of the inventive structures beyond the butyl residue has not been disclosed in the prior art. The sum of the above-mentioned differences between the well-known compounds and those according to the invention results in a significant specificity of the molecules in accordance with general formulas (1) to (16) when regarding the treatment of tumors, especially tumors of the skin. For example, similar compounds of the prior art have an effect on a variety of polymerase, whereas the structures according to the invention are especially efficient on polymerase alpha. The well-known prior art compounds, which have an effect e.g. on a number of polymerases, do not have a specific effect on tumors, especially tumors of the skin.

In a preferred embodiment of the invention the pharmaceutical carrier is selected from the group comprising fillers, diluents, binders, humectants, dissolution retarders, disintegrants, absorption enhancers, wetting agents, adsorbents, lubricants and/or carrier lipids, especially solid lipid nanoparticles, nano-structured lipid carriers, liposomes as well as polymer particles, especially dendrimers. A pharmaceutical agent in the meaning of the invention is any agent in the field of medicine, which can be used in the prophylaxis, diagnosis, therapy, follow-up or aftercare of patients who have come in contact particularly with tumor cells or cancerogens in such a way that a pathogenic modification of the overall condition or of the condition of particular parts of the organism could establish at least temporarily. Thus, for example, the pharmaceutical agent in the meaning of the invention can be a vaccine, an immunotherapeutic or immunoprophylactic agent. The pharmaceutical agent in the meaning of the invention may comprise the compound of the invention or the compound of the invention and/or an acceptable salt or components thereof.

The invention also relates to a kit comprising the pharmaceutical agent of the invention together with an application system as well as information for combining the contents of the kit. The information for combining the contents of the kit relates to the use of said kit in the prophylaxis and/or therapy of diseases, particularly tumoral diseases. For example, the information may also concern a therapeutic regime, i.e. a concrete injection or application schedule, the dose to be administered, or other things.

In a further aspect the invention relates to the use of the agents of the invention, the pharmaceutical agents of the invention and the kit of the invention for the modification of a polymerase in vitro or in vivo.

In a preferred embodiment of the invention the polymerase is a DNA polymerase alpha.

In another preferred embodiment of the invention the modification of a polymerase is modification of cell growth and/or of a cell growth disorder.

In another preferred embodiment of the invention the cell growth disorder is cell proliferation.

The term “tumor” in this invention is intended to encompass all those diseases being described by the term “cancer”. On the other hand, any tissue proliferation or spatial expansion in a human or animal body can be regarded as a tumor in the meaning of the invention, particularly if such spatial expansion or tissue proliferation takes place on an area of the skin. Spatial expansion and tissue proliferation involve swellings, inflammations as well as neoplasms of body tissue as a result of cell growth dysregulation. Accordingly, benign tumors such as birthmarks or fatty swellings are also defined as tumors in the meaning of the invention.

For example, a malignant skin tumor can be a basalioma (basal cell carcinoma), a spinalioma (spinocellular carcinoma) or a malignant melanoma (black skin cancer).

In another preferred embodiment of the invention, cell proliferation is selected from the group comprising skin cancer of epithelial origin, preferably basalioma or spinalioma, skin cancer of pigment cells, skin cancer of immune cells, fibrosarcoma, perspiratory gland carcinoma, sebaceous gland carcinoma, angiosarcoma, myosarcoma and/or Merkel cell carcinoma.

In another preferred embodiment of the invention the basalioma is a nodular, solid basalioma, a superficial basalioma, a pigmented basalioma, a sclerotizing basalioma, an exulcerating basalioma and/or a destructive basalioma.

In another preferred embodiment of the invention the cell growth disorder is a keratosis.

In another preferred embodiment of the invention the keratosis is selected from the group comprising seborrhoeic keratosis, actinic keratosis, senile speckles, pigmentation marks and/or lentigo solaris.

In yet another preferred embodiment of the invention the tumor disease is a carcinoma, a sarcoma, a neuroendocrine tumor, a hemooncologic tumor, a dysontogenetic tumor and/or a mixed tumor.

In still another preferred embodiment the disease is a cancerous disease that is treated, prophylactically prevented, or whose recurrence is prevented, selected from the group of cancerous diseases or tumor diseases of the ear-nose-throat region, of the lungs, mediastinum, gastrointestinal tract, urogenital system, gynecological system, breast, endocrine system, skin, bone and soft-tissue sarcomas, mesotheliomas, melanomas, neoplasms of the central nervous system, cancerous diseases or tumor diseases during infancy, lymphomas, leukemias, paraneoplastic syndromes, metastases with unknown primary tumor (CUP syndrome), peritoneal carcinomatoses, immunosuppression-related malignancies and/or tumor metastases.

More specifically, the tumors can be the following types of cancer: adenocarcinoma of breast, prostate and colon; all forms of lung cancer starting in the bronchial tube; bone marrow cancer, melanoma, hepatoma, neuroblastoma; papilloma; apudoma, choristoma, branchioma; malignant carcinoid syndrome; carcinoid heart disease, carcinoma (for example, Walker carcinoma, basal cell carcinoma, squamobasal carcinoma, Brown-Pearce carcinoma, ductal carcinoma, Ehrlich tumor, in situ carcinoma, cancer-2 carcinoma, Merkel cell carcinoma, mucous cancer, non-parvicellular bronchial carcinoma, oat-cell carcinoma, papillary carcinoma, scirrhus carcinoma, bronchio-alveolar carcinoma, bronchial carcinoma, squamous cell carcinoma and transitional cell carcinoma); histiocytic functional disorder; leukemia (e.g. in connection with B cell leukemia, mixed-cell leukemia, null cell leukemia, T cell leukemia, chronic T cell leukemia, HTLV-II-associated leukemia, acute lymphocytic leukemia, chronic lymphocytic leukemia, mast cell leukemia, and myeloid leukemia); malignant histiocytosis, Hodgkin's disease, non-Hodgkin lymphoma, solitary plasma cell tumor; reticuloendotheliosis, chondroblastoma; chondroma, chondrosarcoma; fibroma; fibrosarcoma; giant cell tumors; histiocytoma; lipoma; liposarcoma; leukosarcoma; mesothelioma; myxoma; myxosarcoma; osteoma; osteosarcoma; Ewing sarcoma; synovioma; adenofibroma; adenolymphoma; carcinosarcoma, chordoma, craniopharyngioma, dysgerminoma, hamartoma; mesenchymoma; mesonephroma, myosarcoma, ameloblastoma, cementoma; odontoma; teratoma; thymoma, chorioblastoma; adenocarcinoma, adenoma; cholangioma; cholesteatoma; cylindroma; cystadenocarcinoma, cystadenoma; granulosa cell tumor; gynadroblastoma; hidradenoma; islet-cell tumor; Leydig cell tumor; papilloma; Sertoli cell tumor, theca cell tumor, leiomyoma; leiomyosarcoma; myoblastoma; myoma; myosarcoma; rhabdomyoma; rhabdomyosarcoma; ependymoma; ganglioneuroma, glioma; medulloblastoma, meningioma; neurilemmoma; neuroblastoma; neuroepithelioma, neurofibroma, neuroma, paraganglioma, non-chromaffin paraganglioma, angiokeratoma, angiolymphoid hyperplasia with eosinophilia; sclerotizing angioma; angiomatosis; glomangioma; hemangioendothelioma; hemangioma; hemangiopericytoma, hemangiosarcoma; lymphangioma, lymphangiomyoma, lymphangiosarcoma; pinealoma; cystosarcoma phylloides; hemangiosarcoma; lymphangiosarcoma; myxosarcoma, ovarian carcinoma; sarcoma (for example, Ewing sarcoma, experimentally, Kaposi sarcoma and mast cell sarcoma); neoplasms (for example, bone neoplasms, breast neoplasms, neoplasms of the digestive system, colorectal neoplasms, liver neoplasms, pancreas neoplasms, hypophysis neoplasms, testicle neoplasms, orbital neoplasms, neoplasms of the head and neck, of the central nervous system, neoplasms of the hearing organ, pelvis, respiratory tract and urogenital tract); neurofibromatosis and cervical squamous cell dysplasia.

In another preferred embodiment the cancerous disease or tumor being treated or prophylactically prevented, or whose recurrence is prevented, is selected from the following group of cancerous diseases or tumor diseases: tumors of the ear-nose-throat region, comprising tumors of the inner nose, nasal sinus, nasopharynx, lips, oral cavity, oropharynx, larynx, hypopharynx, ear, salivary glands, and paragangliomas, tumors of the lungs, comprising non-parvicellular bronchial carcinomas, parvicellular bronchial carcinomas, tumors of the mediastinum, tumors of the gastrointestinal tract, comprising tumors of the esophagus, stomach, pancreas, liver, gallbladder and biliary tract, small intestine, colon and rectal carcinomas and anal carcinomas, urogenital tumors comprising tumors of the kidneys, ureter, bladder, prostate gland, urethra, penis and testicles, gynecological tumors comprising tumors of the cervix, vagina, vulva, uterine cancer, malignant trophoblast disease, ovarian carcinoma, tumors of the uterine tube (Tuba Faloppii), tumors of the abdominal cavity, mammary carcinomas, tumors of the endocrine organs, comprising tumors of the thyroid, parathyroid, adrenal cortex, endocrine pancreas tumors, carcinoid tumors and carcinoid syndrome, multiple endocrine neoplasias, bone and soft-tissue sarcomas, mesotheliomas, skin tumors, melanomas comprising cutaneous and intraocular melanomas, tumors of the central nervous system, tumors during infancy, comprising retinoblastoma, Wilms tumor, neurofibromatosis, neuroblastoma, Ewing sarcoma tumor family, rhabdomyosarcoma, lymphomas comprising non-Hodgkin lymphomas, cutaneous T cell lymphomas, primary lymphomas of the central nervous system, Hodgkin's disease, leukemias comprising acute leukemias, chronic myeloid and lymphatic leukemias, plasma cell neoplasms, myelodysplasia syndromes, paraneoplastic syndromes, metastases with unknown primary tumor (CUP syndrome), peritoneal carcinomatosis, immunosuppression-related malignancy comprising AIDS-related malignancies such as Kaposi sarcoma, AIDS-associated lymphomas, AIDS-associated lymphomas of the central nervous system, AIDS-associated Hodgkin disease, and AIDS-associated anogenital tumors, transplantation-related malignancy, metastasized tumors comprising brain metastases, lung metastases, liver metastases, bone metastases, pleural and pericardial metastases, and malignant ascites.

In another preferred embodiment the cancerous disease or tumor being treated or prophylactically prevented, or whose reappearance is prevented, is selected from the group comprising cancerous diseases or tumor diseases such as mammary carcinomas, gastrointestinal tumors, including colon carcinomas, stomach carcinomas, large intestine cancer and small intestine cancer, pancreas carcinomas, ovarian carcinomas, liver carcinomas, lung cancer, renal cell carcinomas, multiple myelomas.

In a particularly preferred fashion the agents according to the invention are intended for topical and systemic treatment of breast, colon, rectum and urinary bladder.

Furthermore, the agents according to the invention can be used for the treatment of keloids and plaque psoriasis.

In another preferred embodiment of the invention the agent is in the form of a solution, emulsion, suspension, ointment, balm, oil, gel, foam, eye balm, eye gel, suppository, spray, pad, stick or crayon, in liquid form, in the form of artificial tears, thermoreversible gel (to be used in liquid form), in the form of a tampon containing active substance and/or in the form of a cream. It was completely surprising to find that the advantages of the agent according to the invention can be even more improved by incorporating the agent in the above-mentioned galenic forms. A person skilled in the art will be familiar with other formulation concepts of incorporating the inventive agents in vehicles such as emulsions or other products for dermal application such as liquid forms which preferably can be free of water or may contain water, and those containing water can be divided into single-phase systems and multi-phase systems according to the invention. Furthermore, semi-solid forms can be used, which can be free of water or may contain water and can likewise be divided into single-phase systems and multi-phase systems. Furthermore, solid forms that are lipophilic or hydrophilic can be used in a preferred manner. In addition to those mentioned above, examples of such forms are e.g. fatty ointments, foams, powders, crayons, gel creams, hydrodispersion gels, fluid emulsions, lotions, ointments, sprays and creams. As is well-known to those skilled in the art, such vehicles can be differentiated into rich/valuable and fresh/light with respect to their skin feel on the one hand and, with respect to their viscosity on the other hand, into those having low viscosity and those having high viscosity. Nanoemulsions or oils or oleo-gels tend to have low viscosity, whereas hydrogels or hydrocreams or O/W emulsions or W/O emulsions have high viscosity. If liquid administration forms are used, they can be divided into water-free and water-containing systems. In particular, non-polar systems, polar systems with no emulsifier and polar systems including an emulsifier are preferred among systems free of water. Single-phase systems such as solutions and microemulsions are preferred among systems containing water, and multiple emulsions, W/O emulsions or O/W emulsions are preferred among multi-phase systems. Among solid/liquid systems, preferred forms are suspensions or liquid/solid/liquid systems such as suspension/emulsion systems. Those skilled in the art will be familiar with various ways of providing such pharmaceutical carriers. Among O/W emulsions, preferred galenic lead substances are O/W emulsifiers, W/O emulsifiers, liquid hydrophilic components and liquid lipophilic components. Among W/O emulsions, preferred galenic lead substances are W/O emulsifiers, O/W emulsifiers, liquid and semi-solid lipophilic components, gelling agents, liquid hydrophilic components and/or salts.

Among the semi-solid preferred vehicles, water-free systems as well as water-containing systems are preferred for different uses. Systems free of water can be constituted of non-polar systems or of polar systems with no emulsifiers, such as lipogels, oleo-gels or polyethylene glycol gels, or of non-polar systems including an emulsifier on O/W absorption bases or W/O absorption bases. In a preferred fashion the water-containing systems can be constituted of single-phase systems, such as hydrogels or microemulsion gels, or multi-phase systems, such as O/W creams, W/O creams, or amphiphilic systems. In a temperature range between room temperature and skin temperature the preferred semi-solid preparations are spreadable preparations for use on the skin or mucosa, which have a local effect, transport active substances, or have an emollient or protective effect on the skin. Preferred preparations are ointments in a stricter sense, creams, gels and/or pastes. In addition to ointments, creams, gels and pastes, it is also possible to use oleo-gels in the form of semi-solid, transparent single-phase systems. From the U.S. Pat. No. 6,187,323 or from Aiache et al., 2001, for example, various water-free compounds for the formulation of semi-solid systems are known to those skilled in the art, e.g. a combination of an oleo-gel and a hydrogel, which, according to the invention, can be referred to as bi-gel. Furthermore, hydrodispersion gels or various lipids can be used to provide the vehicles according to the invention. When using lipids, it is possible to employ organosilicon compounds as well as carbon-organic compounds to provide lipid phases in dispersed systems, in which case carbon-organic compounds can be provided using, for example, non-hydrolyzable lipids or hydrolyzable lipids (glycerols), wax esters. The advantages of such systems are improved smoothness and enhanced elasticity of the skin and—depending on the composition of the lipids—their ability to furnish an influence on liberation and penetration. It is well-known to a person skilled in the art which lipids are to be used in order to, for example, increase or reduce the penetration within a time parameter.

Other preferred vehicles are, for example, hydrodispersion gels or microcapsules, microspherules or pellets (macrobeads). The above-mentioned carriers are used to increase the stability and ensure a minimum application time on the skin. The preferred semi-solid single-phase systems can be prepared using the following galenic lead substances: liquid hydrophilic components, especially water and (poly)alcohols, hydrophilic gel-forming agents, salt-forming substances, as well as W/O emulsifiers, O/W emulsifiers, liquid, semi-solid and solid lipophilic components as well as lipophilic gel-forming agents or builders. It is well-known to those skilled in the art in which way these substances must be combined in order to achieve a specific effect.

Other galenic preparations for dermal products are well-known to those skilled in the art. According to the present application, it is possible to use e.g. all those galenic combinations disclosed by Daniel and Knie in JDDG; 2007, 5: 367-383. It is well-known to those skilled in the art that different galenic preparations have different effects on the skin and incorporate the inventive agents in the skin to different extents. The contents of JDDG; 2007, 5: 367-383, is hereby incorporated in the disclosure of the teaching according to the invention. Preferred products according to the invention are, for example, lipophilic or hydrophilic solutions, lipophilic or hydrophilic emulsions, lipophilic or hydrophilic suspensions, special liquid preparations, hydrophobic or hydrophilic ointments, water-emulsifying ointments, lipophilic, hydrophilic or amphiphilic creams, hydrogels, hydrophobic or hydrophilic pastes and/or powders.

In a particularly advantageous manner it is also possible to use nanoscale transport systems having a dendritic architecture, such as disclosed in DE 10 2004 039 875, which hereby is incorporated in the disclosure of the teaching according to the present application. A variety of transport systems are known from the prior art, such as liposomes, polymer micelles, polymer conjugates or simple dendritic core-shell architectures. Polymer micelles are physical aggregates of ambiphilic macromolecules, which can form spontaneously in water via self-organization. In general, the inner block is non-polar or ionic and the outer block protecting the core via steric stabilization is polar. Frequently, they are used to solubilize non-polar active substances or combinations of active substances with limited solubility in water or to transport oligonucleotides. Such dermal transport systems can be used with advantage to facilitate rapid passage of the agent according to the invention into tumors.

Nanoscale transport systems with a simple dendritic core-shell architecture likewise facilitate the transport of active substances. In contrast to physical aggregates of ambiphilic molecules, covalent modification of dendritic macromolecules with a corresponding shell can produce stable micellar structures suitable for encapsulation of medical substances. Even more advantageously, nanoscale transport systems constituted of at least one dendritic core and at least two shells can be used. The shells preferably have different polarities so that a polarity gradient is achieved which is capable of enclosing both non-polar and polar active substances or combinations of active substances. Advantageous nanoscale transport systems therefore have a multi-shell unimolecular structure. By combining different shells it is very well possible to constantly create new nanoscale transport systems adapted to the active substance and the use thereof. Dendrimers as well as hyperbranched polymers can be used for such a nanoscale transport system. Consequently, the use of hyperbranched polymers is also advantageous because it is possible in this way to reduce both synthetic efforts and expenses. In a preferred embodiment the above-mentioned dendritic core consists of polyglycerol, polyamide, polyamine, polyether or polyester. These compounds can be further modified within the dendritic architecture. As a consequence, the dendritic core can be polarized according to the modification thereof. It was completely surprising to find that such agents are particularly suitable for application through the skin. Compared to other lipid nanoparticles, they are about four times as suitable when application of particles into the skin is concerned.

Another transdermal therapeutic system with increased flux of active substance has been disclosed in DE 10 2006 026 578, which is likewise incorporated in the disclosure of the present application. This likewise preferred transdermal therapeutic system can be employed in the form of a transdermal pad having an active substance-containing matrix of agents according to the invention. For example, the matrix can be made of rubber or other synthetic polymers having water-soluble or water-swellable inclusion bodies embedded therein, which contain micronized or nanoscale active substance particles comprising the agents according to the invention. Compared to other prior art agents, this transdermal therapeutic system can achieve improved stability and improved constancy in providing an increased flux of active substance with lower input of adjuvants. It is of course also possible to employ galenic vehicles which themselves have an anti-tumoral effect and can be put to topical use, such as the composition in accordance with DE 03 251 884 (EP 1 348 441), which is likewise incorporated in the disclosure of the teaching according to the present application.

Furthermore, it may be advantageous to provide nano-structured lipid carriers for the dermal applications according to the invention. As a result of the special matrix structure of the nano-structured lipid carriers (NLC), the load capacity can be increased and particle dispersions can be provided which have the consistency of a cream or paste. In particular, the solid lipid matrix of the NLC can protect the inventive pharmaceutical agents from premature degradation. Following dermal application, the NLC form a film which reduces water loss of the skin and can result in increased moisture of the skin, thereby allowing increased penetration of the active substances according to the invention. Also, microcapsules based on liposomes, such as Smarticles and Cagicles, can be used with advantage. Furthermore, the use of colloidal medical forms such as nanoparts, nanocapsules and micelles can be advantageous, in which case stable physiological mixed micelles are concerned which can be used with advantage on particular tumors. However, supportive active substances or pharmacological carriers from the corneotherapy can also be used. This is a so-called outside-in therapy where penetration through the horny layer is advantageous for the primary effect thereof. To this end, the actual active substances of the invention are used e.g. in combination with ceramides, proteins and synthetic analogs thereof, or peptides. Advantageously, such compounds in combination with liposomes and nanoparticles can be adapted to individual preconditions and specific tumors. A person skilled in the art will be familiar with the galenic preconditions of corneotherapy so that application to the teaching of the invention is possible.

In another preferred embodiment of the invention the pharmaceutical agent according to the invention is prepared and used in the form of a gel, poudrage, powder, tablet, sustained-release tablet, premix, emulsion, brew-up formulation, drops, concentrate, granulate, syrup, pellet, bolus, capsule, aerosol, spray and/or inhalant.

In another preferred embodiment of the invention the ointment is selected from the group comprising unguentum leniens (cooling ointment), unguentum emulsificans (hydrophilic ointment), unguentum emulsificans aquosum (water-containing hydrophilic ointment), unguentum cetomacrogolis, unguentum cetylicum cum aqua (cetyl ointment), unguentum alcoholum lanae or unguentum adeps lanae (wool wax alcohol ointment, eucerine), unguentum molle (soft ointment) and/or unguentum zinci (zinc ointment).

In another preferred embodiment of the invention the pharmaceutical agent according to the invention is present in a preparation at a concentration of from 0.1 to 99.5, preferably from 0.5 to 95.0 and more preferably from 20.0 to 80.0 wt. %.

That is, the compounds of the invention are present in the above-specified pharmaceutical formulations, e.g. tablets, pills, granulates and others, at a concentration of preferably 0.1 to 99.5 wt. % of the overall mixture. The amount of active substance, i.e. the amount of an inventive compound combined with the carrier materials to produce a single dosage form, can vary depending on the patient to be treated and on the particular type of administration. Once the condition of a patient has improved, the proportion of active compound in the preparation can be modified so as to obtain a maintenance dose. Depending on the symptoms, the dose or frequency of administration or both can subsequently be reduced to a level where the improved condition is retained. Once the symptoms have been alleviated to the desired level, the treatment should be terminated. However, patients may require an intermittent treatment on a long-term basis if any symptoms of the disease should recur. Accordingly, the proportion of the compounds, i.e. their concentration, in the overall mixture of the pharmaceutical preparation, as well as the composition or combination thereof, is variable and can be modified and adapted by a person of specialized knowledge in the art.

Those skilled in the art will be aware of the fact that the compounds of the invention can be contacted with an organism, preferably a human or an animal, on various routes. Furthermore, a person skilled in the art will also be familiar with the fact that the pharmaceutical agents, in particular, can be applied at varying dosages. Application should be effected in such a way that a tumoral disease is combated as effectively as possible or the onset of such a disease is prevented by prophylactic administration. Concentration and type of application can be determined by a person skilled in the art using routine tests. Apart from dermal application, preferred applications of the compounds of the invention are oral or other application in the form of powders, tablets, fluid mixtures, drops, capsules or the like, rectal application in the form of suppositories, solutions and the like, parenteral application in the form of injections, infusions and solutions, inhalation of vapors, aerosols and powders and pads, and local application in the form of ointments, pads, dressings, lavages and the like. Contacting with the compounds according to the invention is preferably effected in a prophylactic or therapeutic fashion. In prophylactic administration, development of tumors is to be prevented. In therapeutic contacting, a tumor disease is already existing, and the cancer cells already present in the body should be either destroyed or inhibited in their growth. Other forms of application preferred for this purpose are e.g. subcutaneous, sublingual, intravenous, intramuscular, intraperitoneal and/or topical forms.

For example, the suitability of the selected form of application, of the dose, application regimen, selection of adjuvant and the like can be determined by taking serum aliquots from the patient or by using imaging methods in the course of the treatment procedure. Alternatively or concomitantly, the condition of the liver, but also, the amount of T cells or other cells of the immune system can be determined in a conventional manner so as to obtain a general survey on the immuconstitution of the patient and, in particular, the constitution of organs important to the metabolism, particularly of the liver. Additionally, the clinical condition of the patient can be observed for the desired effect, especially antitumoral effect. Tumor diseases can be associated with further infections, e.g. bacterial or mycotic, for which reason additional clinical co-monitoring of the course of such concomitant infections is also possible. Where insufficient antitumoral effectiveness is achieved, the patient can be subjected to further treatment using the agents of the invention, optionally modified with other well-known medicaments expected to bring about an improvement of the overall constitution. Obviously, it is also possible to modify the carriers or vehicles of the pharmaceutical agent or to vary the route of administration. In addition to dermal uptake, e.g. intramuscular or subcutaneous injections or injections into the blood vessels can be envisaged as other preferred routes of therapeutic administration of the compounds according to the invention. At the same time, supply via catheters or surgical tubes can also be used.

Typically, there is an optimum ratio of compound(s) of the invention with respect to each other and/or with respect to other therapeutic or effect-enhancing agents (such as transport inhibitors, metabolic inhibitors, inhibitors of renal excretion or glucuronidation, such as probenecid, acetaminophen, aspirin, lorazepan, cimetidine, ranitidine, colifibrate, indomethacin, ketoprofen, naproxen etc.) where the active substances are present at an optimum ratio. Optimum ratio is defined as the ratio of compound(s) of the invention to other therapeutic agent(s) where the overall therapeutic effect is greater than the sum of the effects of the individual therapeutic agents. In general, the optimum ratio is found when the agents are present at a ratio of from 10:1 to 1:10, from 20:1 to 1:20, from 100:1 to 1:100 and from 500:1 to 1:500.

In some cases, an exceedingly small amount of a therapeutic agent will be sufficient to increase the effect of one or more other agents. In addition, the use of the compounds of the invention in combinations is particularly beneficial to reduce the risk of developing tumor resistance. Of course, the compounds of the invention can be used in combination with other well-known antitumor agents. Such agents are well-known to those skilled in the art. Accordingly, the compounds of the invention can be administered together with all conventional agents, especially other drugs, available for use particularly in connection with tumor drugs, either as a single drug or in a combination of drugs. They can be administered alone or in combination with same.

In a preferred fashion the compounds of the invention are administered together with said other well-known pharmaceutical agents at a ratio of about 0.005 to 1. Preferably, the compounds of the invention are administered particularly together with tumor-inhibiting agents at a ratio of from 0.05 to about 0.5 parts and up to about 1 part of said known agents. In this event, antibacterial agents can also be concerned. The pharmaceutical composition can be present in substance or as an aqueous solution together with other materials such as preservatives, buffer substances, agents to adjust the osmolarity of the solution, and so forth.

The utilization of endocytosis for the cellular uptake of antitumoral compounds is highly effective for some, particularly long-lived, substances, but is very difficult to transfer to more general uses. One alternative is the prodrug concept generally known to those skilled in the art. By definition, a prodrug includes its active substance in the form of a non-active precursor metabolite. It is possible to distinguish between carrier prodrug systems and biotransformation systems. The latter include the active substance in a form requiring chemical or biological metabolization. Such prodrug systems are well-known to those skilled in the art. Carrier prodrug systems include the active substance as such, bound to a masking group which can be cleaved off by a preferably simple controllable mechanism. The inventive function of masking groups in the compounds of the invention is neutralization of the charge for improved reception by cells. When using the compounds of the invention together with a masking group, the latter may also influence other pharmacological parameters, such as oral bioavailability, distribution in tissue, pharmacokinetics, as well as stability to non-specific phosphatases. In addition, delayed release of the active substance may entail a depot effect. Furthermore, modified metabolization may occur, thereby achieving higher efficiency of the active substance or organ specificity. In the event of a prodrug formulation, the masking group, or a linker group binding the masking group to the active substance, is selected in such a way that the prodrug has sufficient hydrophilicity to be dissolved in the blood serum, sufficient chemical and enzymatic stability to reach the site of action, and hydrophilicity suitable for diffusion-controlled membrane transport. Furthermore, it should permit chemical or enzymatic liberation of the active substance within a reasonable period of time and, of course, the liberated auxiliary components should not be toxic. In the meaning of the invention, however, the compound with no mask or no linker and no mask can also be understood as prodrug which initially must be produced via enzymatic and biochemical processes from the incorporated compound in the cell.

In another preferred embodiment of the invention the pharmaceutical agent according to the invention is employed in a total amount of from 0.05 to 500 mg per kg, preferably from 5 to 100 mg per kg body weight per 24 hours.

In another preferred embodiment of the invention the preparation is employed orally, subcutaneously, intravenously, intramuscularly, intraperitoneally and/or topically.

In another preferred embodiment of the invention the agents according to the invention, the pharmaceutical agents of the invention and the kit of the invention are used in secondary prophylaxis of tumors.

The invention also relates to the use of the inventive compounds or the inventive molecules in accordance with general formulas 1 to 16 as lead structures for the development of polymerase inhibitors.

The teaching according to the present application is remarkable for the following features:
Departure from conventional technologies
New field of problems
Existence of a long-unsatisfied, urgent need for the solution of the problem solved by the invention
Hitherto vain efforts in the art
Simplicity of a particular solution indicates inventive activity, especially as it replaces more complicated teachings
Development in scientific technology has proceeded in a different direction
Achievement that rationalizes development
Erroneous ideas in the art on the solution of the problem at issue (prejudice)
Technical progress, e.g. improvement, performance enhancement, lower expense, savings of time, materials, work steps, cost or raw materials difficult to obtain, enhanced reliability, elimination of flaws, superior quality, maintenance freedom, greater efficiency, higher yield, expansion of the technical scope, provision of a further means, creation of a second approach, creation of a new field, first-time solution of a problem, reserve means, alternatives, scope for rationalization, automation and miniaturization, or enrichment of the range of available drugs
Fortunate choice out of a variety of possibilities because one has been selected, the result of which has not been predictable, this therefore being a patentable fortunate choice
Errors in the technical literature or highly contradictory representation of the subject matter of the invention
Young field of technology
Combination invention, i.e., several known elements have been combined to achieve a surprising effect
Issue of licenses
Praise in the art
Economic success.

More specifically, the advantageous embodiments of the invention have at least one or more of the above-mentioned advantages.

Without intending to be limiting, the invention will be explained in more detail with reference to an example.

### EXAMPLE

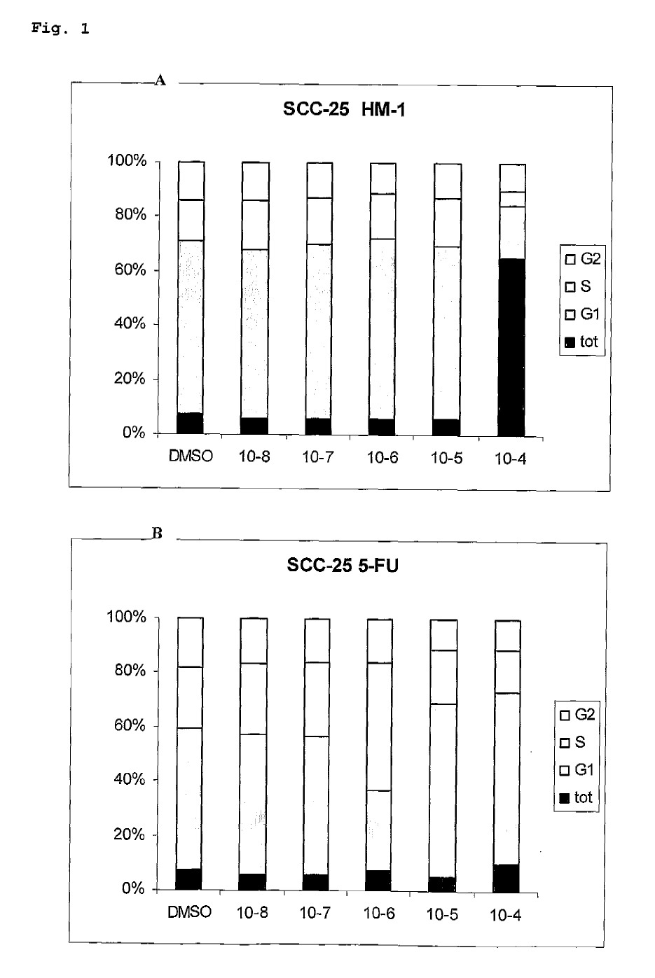

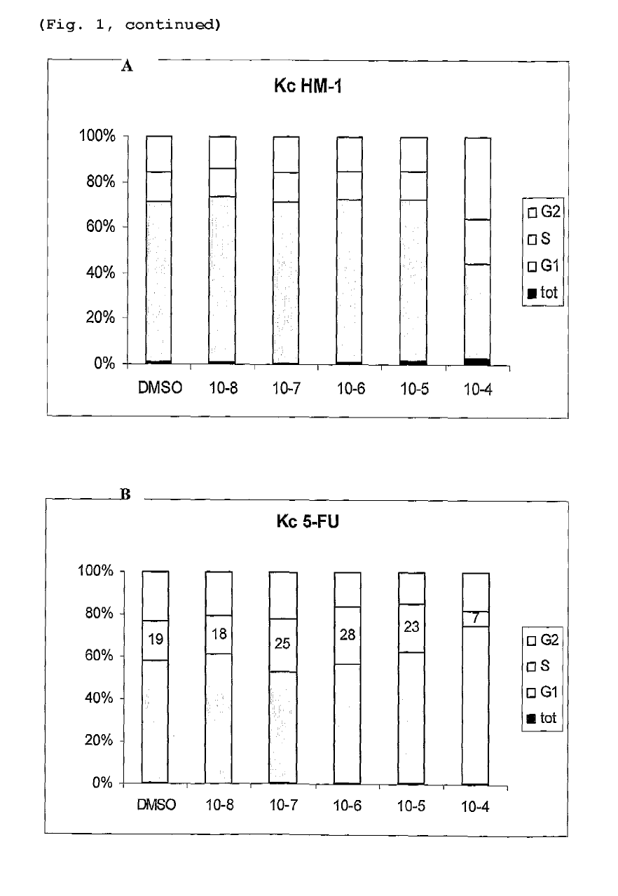

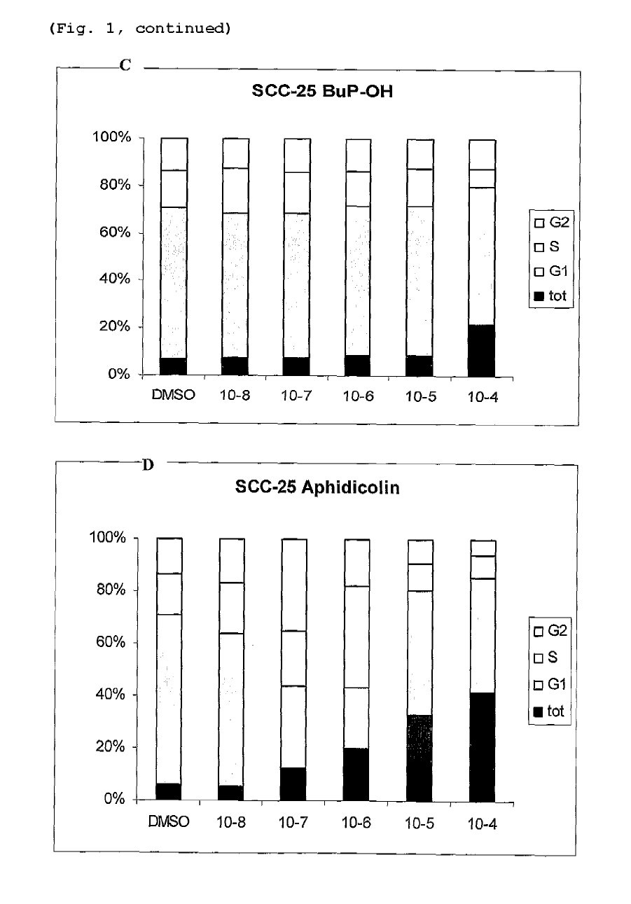

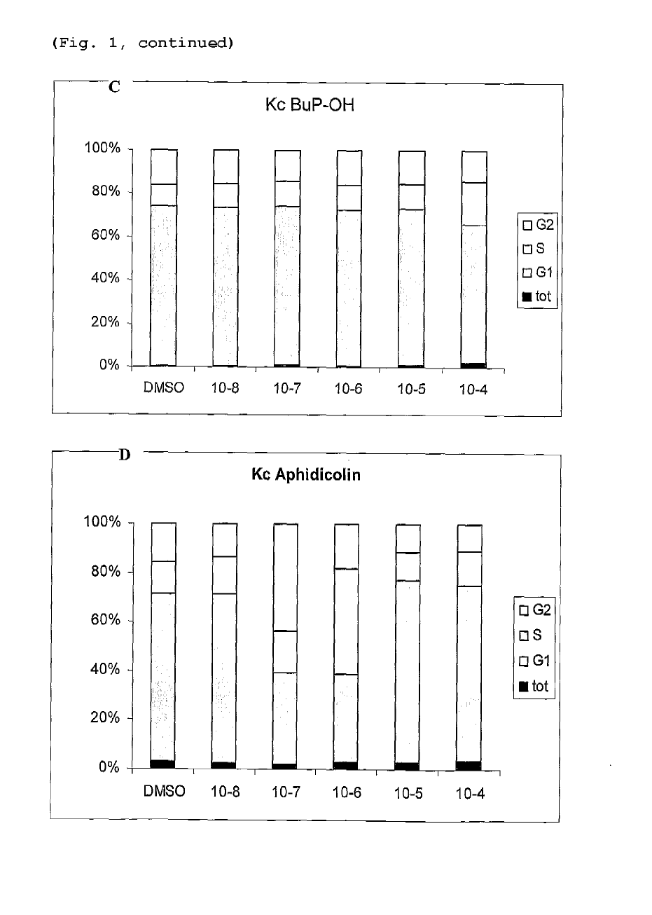

Substance 10 (HM-1) is remarkable for its strong cytotoxic effect on non-contact-inhibited (FIG. 1) and contact-inhibited (Table 1) tumor cells (SCC-25 cells), which is stronger than that in primary keratinocytes (Kc). This results in a selective damage of the skin tumor.

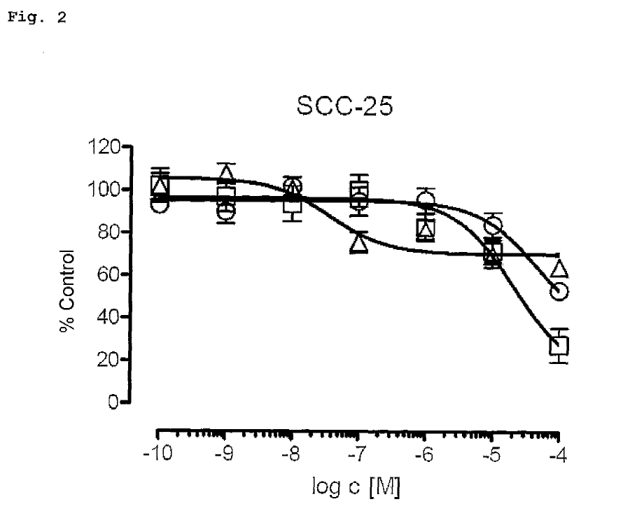

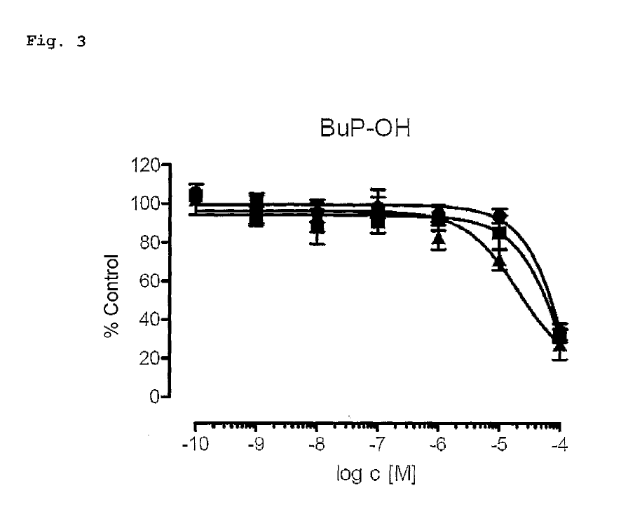

One special advantage of BuP—OH to be mentioned is the particularly strong antiproliferative effect on tumor cells (SCC-25), which surpasses that of HM-1 and HM-1 oxime (FIG. 2). There is some selectivity for tumor cells, and the effect is stronger than that on primary keratinocytes and HaCaT cells (FIG. 3, Table 1).

TABLE 1
−lg IC₅₀ values [M] ± SEM determined from the MTT test
(48 h stimulation). The maximum inhibition was calculated as
inhibitionₘₐₓ = 100 − E; E = activity at 10⁻⁴ M in %.

| | | Primary | SCC-25 |
|---|---|---|---|
| MTT test 48 h | | keratinocytes | cells |
| Aphidicolin | −lg IC₅₀ [M] ± SEM | 8.25 ± 0.40 | 6.29 ± 0.29 |
| (positive control) | max. inhibition | 46% | 66% |
| 5-FU | −lg IC₅₀ [M] ± SEM | 4.98 ± 0.56 | 5.98 ± 0.28 |
| (positive control) | max. inhibition | 44% | 42% |
| BuP—OH | −lg IC₅₀ [M] ± SEM | 3.29 ± 1.74 | 4.69 ± 0.26 |
| | max. inhibition | 66% | 73% |
| HM-1 | −lg IC₅₀ [M] ± SEM | n.d. | 4.35 ± 0.43 |
| | max. inhibition | 24% | 47% |
| HM-1 oxime | −lg IC₅₀ [M] ± SEM | 7.51 ± 0.79 | 7.43 ± 0.32 |
| | max. inhibition | 7% | 36% |
| Foscamet | −lg IC₅₀ [M] ± SEM | 15.97 | 5.87 ± 0.24 |
| (negative control) | max. inhibition | n.d. (−13%) | 9% |

n.d. = not determinable.

The tested active substances at specific concentrations inhibit tumor cells of non-cutaneous origin at low concentrations more strongly than SCC-25 and SCC-4 cells. This applies to e.g. the breast cancer cell line MCF-7 and the colon carcinoma line HT-29. Furthermore, when increasing the concentration of the tested active substances, a good effect on skin cancers, particularly nonmelanoma skin cancer, was found. By varying the concentration, a person skilled in the art can achieve an effect on all claimed tumors in in vitro and in vivo tests. The results presented herein merely represent a detail view on tests at a defined concentration. As is well-known to a person skilled in the art, the concentration of pharmaceutical active substances can be varied at will within specific ranges, depending on the disease, the age and condition of the patient. If an agent fails to show an effect at a particular concentration, a person skilled in the art can routinely test new vehicles or apply the agent several times or within shorter time intervals.

The high selectivity of guanosine phosphonate analogs having only a partial sugar structure is of supreme interest. The members of the substance group 8 are remarkable not only for their strong inhibiting effect on tumor cells but also for the absence of any inhibiting effect on normal keratinocytes (NHK) and the HaCaT keratinocyte cell line. Also, molecular modeling calculations performed on these substances in parallel indicated a particularly high affinity to the binding site. Advantageously, it was found that substances which also may have a cytotoxic effect on dermal fibroblasts can be modified by the preferred carrier systems in such a way that the toxic effect is reduced when the carrier systems used are those targeted to an accumulation of the medical substance in the epidermis.

TABLE 2
Maximum decrease in viability, determined in
the MTT test when incubating with test substances
(selection) at a concentration of 10⁻⁴ M.

| Max. | | | | | | |
|---|---|---|---|---|---|---|
| inhibition (%) | NHK | HaCaT | SCC-25 | Fibrob. | Hat 29 | MCF-7 |
| Aphidicolin | 44 | 86 | 62 | 54 | 70 | 61 |
| (positive control) | | | | | | |
| 5-FU | 41 | 80 | 42 | 88 | 66 | 71 |
| (positive control) | | | | | | |
| Foscamet-Na | 0 | — | 9 | — | — | — |
| (negative control) | | | | | | |
| BuP—OH (6) | 66 | 68 | 65 | 53 | 67 | 77 |
| IsoHex (6 analog) | 46 | — | 54 | — | — | — |
| HM-1 (5) | 23 | 16 | 47 | — | — | — |
| HM-1 oxime | 3 | 20 | 30 | — | — | — |
| (5 derivative) | | | | | | |
| OxBu (8) | 0 | 0 | 30 | 62 | 68 | 62 |
| OxEt (8 analog) | 0 | 0 | 19 | — | — | — |

(—: not investigated)

Carrier Systems

Compared to a conventional cream, solid lipid nanoparticles (150-170 nm) and dendritic core-shell nanotransporters (20-30 nm) increase the epidermal penetration by 3-6 times and 8-13 times, respectively. The penetration-promoting effect results from the favorable ratio of surface (=contact area) to volume, and in addition, lipid particles mix with epidermal lipids so as to promote skin penetration.

The log P values of the guanosine analogs (group 6) and guanosine phosphonate analogs (group 8) are in the same order of magnitude as those of glucocorticoids (prednisolone, prednicarbate), particularly those of prednicarbate which can be stably integrated for a long time (see Table 3, [C. Santos Maia, W. Mehnert, M. Schaller, H. C. Korting, A. Gysler, A. Haberland, M. Schafer-Korting, Drug targeting by solid lipid nanoparticles for dermal use. J Drug Targeting 10 (2002) 489-4951]), and therefore offer surprisingly good preconditions for the preparation of lipid nanoparticles of high stability. Accordingly, they are particularly suitable for the production of drugs.

TABLE 3
logP values of polymerase inhibitors and selected glucocorticoids.

| Active substance | logP |
|---|---|
| HM-1 | −1.13 |
| OxBu | 1.36 |
| OxHe | 2.34 |
| Prednisolone | 1.69 |
| Prednicarbate | 3.82 |

The figures illustrate the following issues:

FIG. 1: Effect of substance 10 (HM-1) and BuPOH on the tumor cell line SCC-25 and primary keratinocytes isolated from foreskin. The effect of HM-1 on SCC-25 cells is stronger than that of the two comparative substances 5-fluorouracil and aphidicolin.

FIG. 2: Viability (MTT test) after 48 h exposure of SCC-25 cells to BuP—OH (□), HM-1 [symbol] and HM-1 oxime (Δ).

FIG. 3: Viability (MTT test) of HaCaT cells (▪), keratinocytes () and SCC-25 cells (▴) after 48 h exposure to BuP—OH.

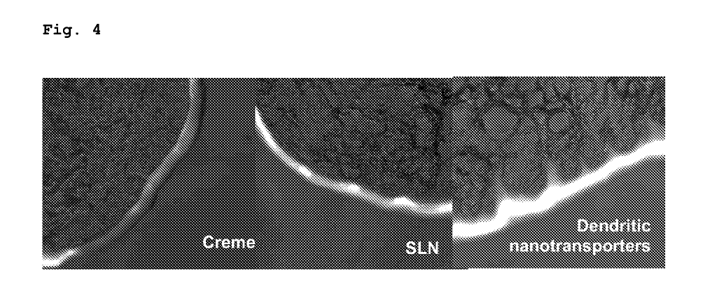

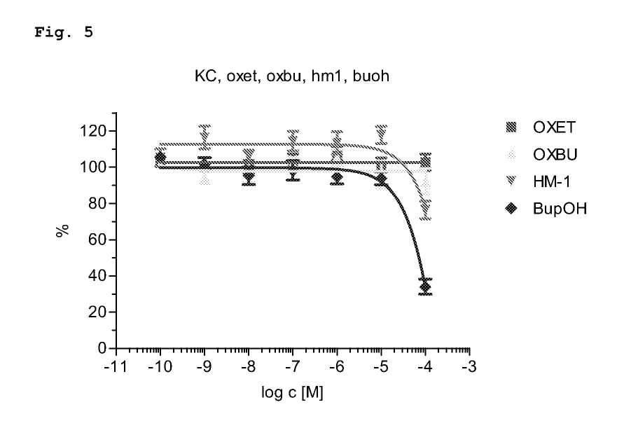

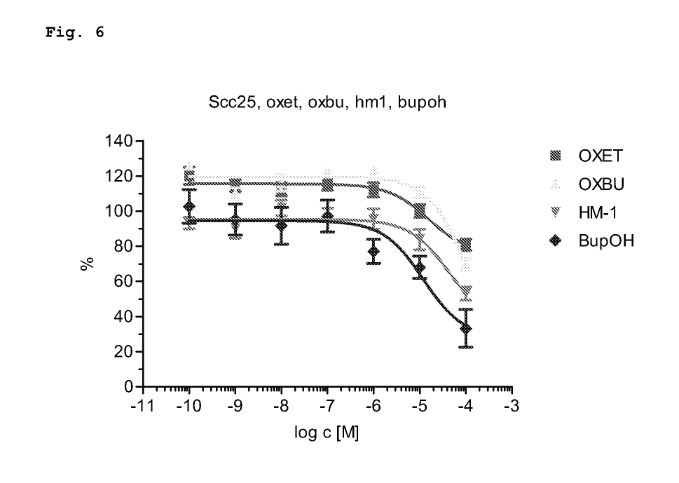

FIG. 4: Penetration enhancement of the model substance Nile red when loading solid lipid nanoparticles (SLN) and dendritic core-shell nanotransporters, respectively, compared to a cream; time of application: 6 h.

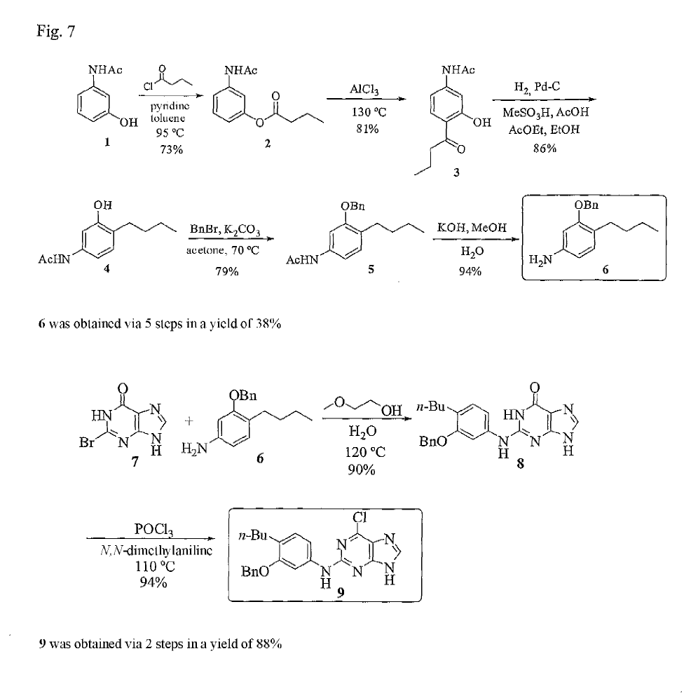

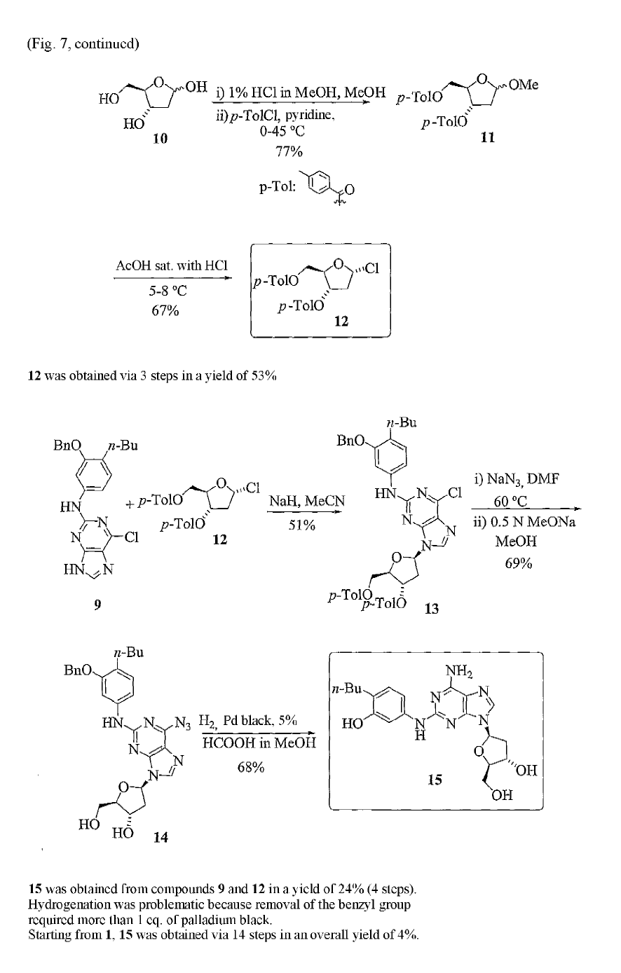

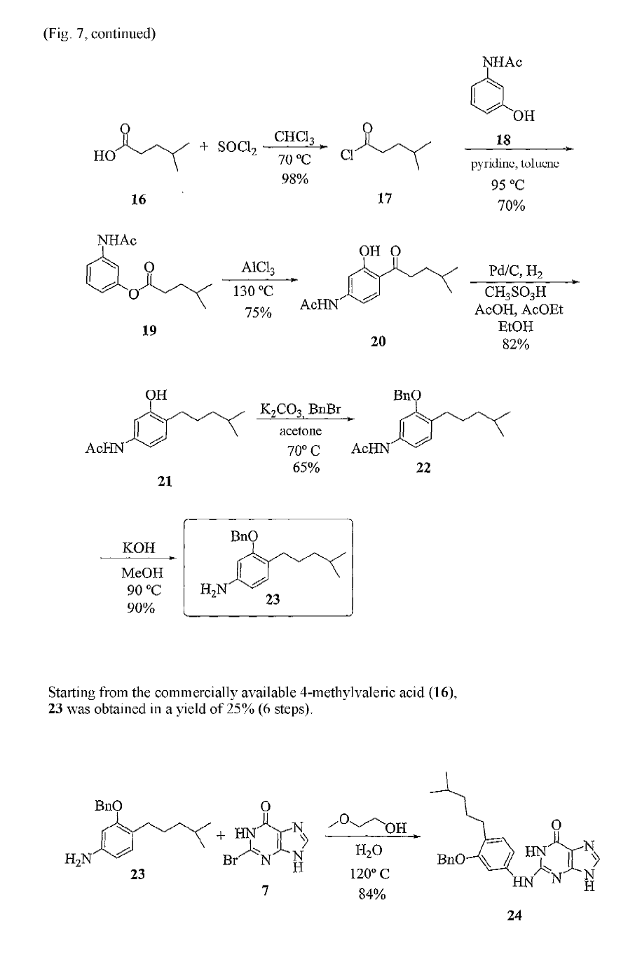

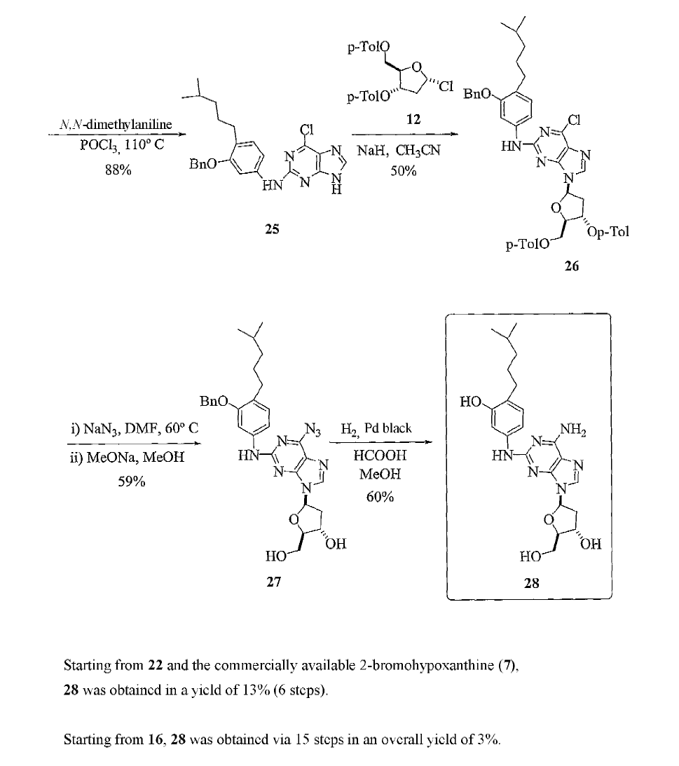

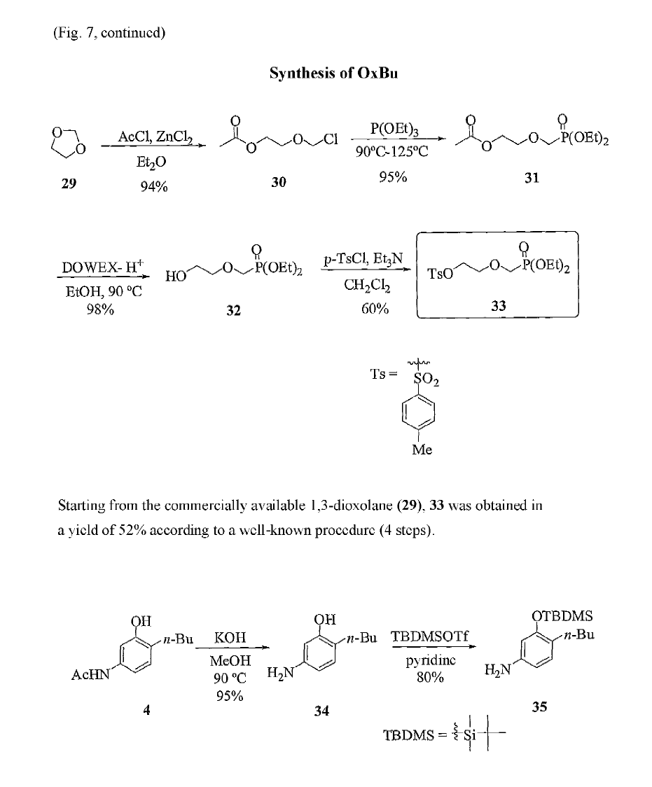

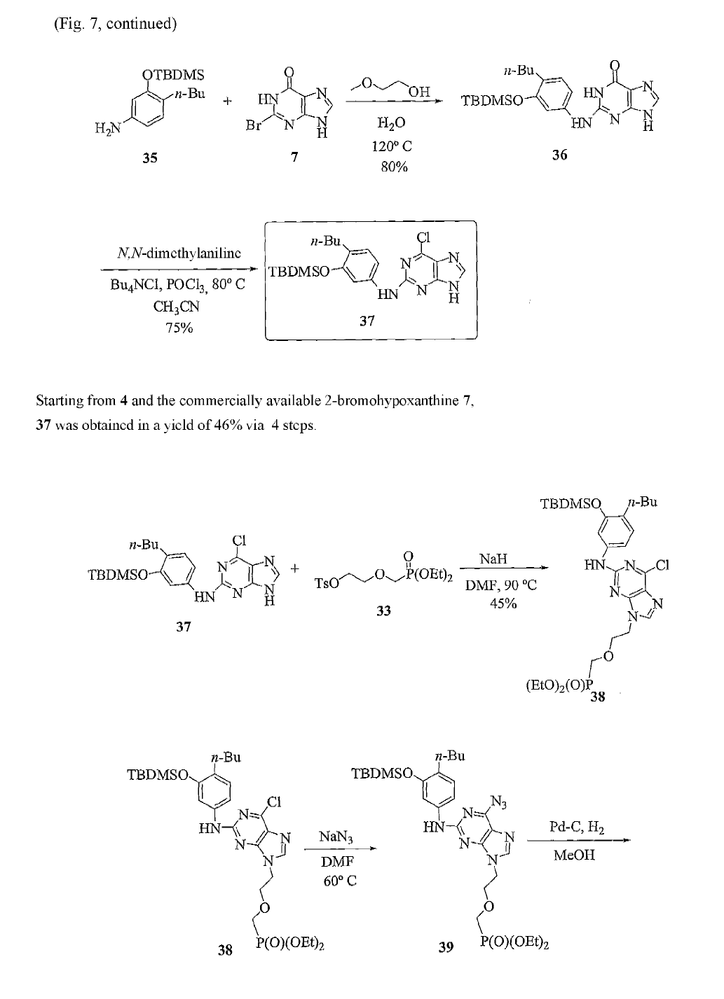

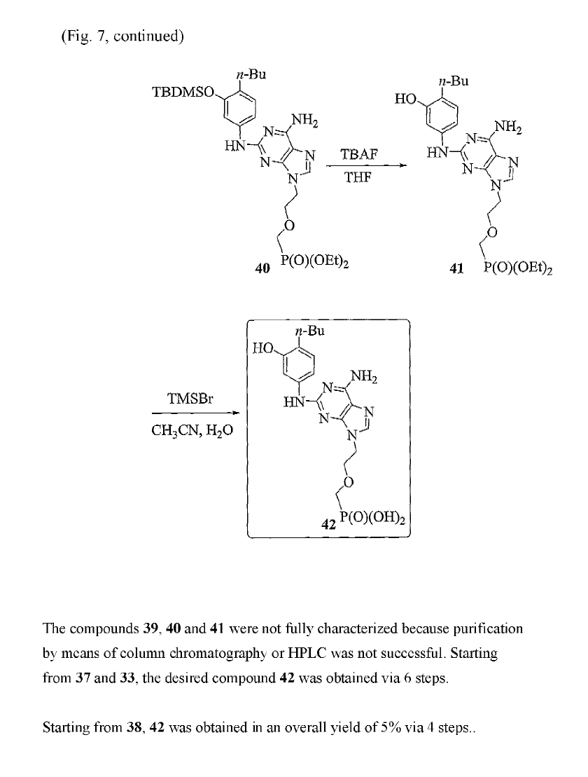

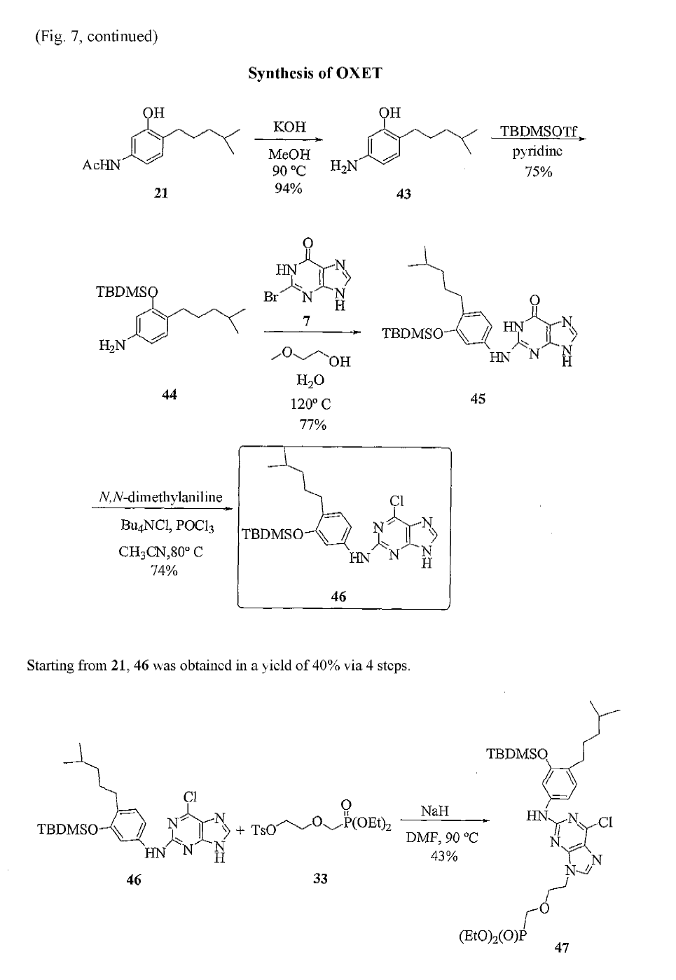

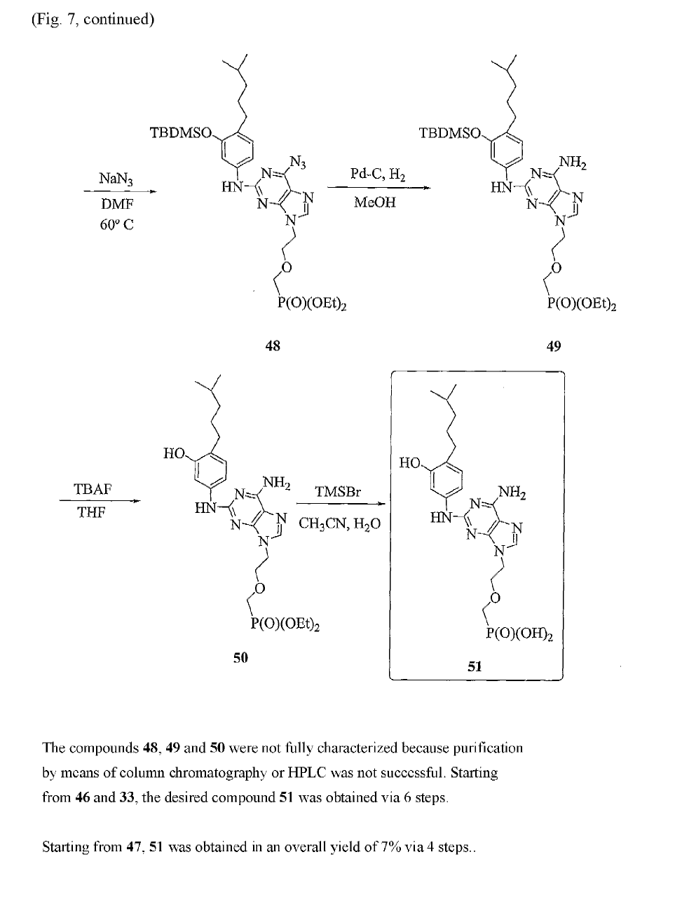

The preparation or synthesis of the inventive antitumor agents for topical use is shown in FIG. 7. For example, the synthesized products 6, 9, 12, 15, 23, 28, 33, 37, 42, 46 and/or 51 are particularly suitable for topical dermal application as antitumor agents.

1. An antitumor agent, especially for the treatment of dermal tumors, selected from the group comprising compounds according to the general formulas (1) to (16): 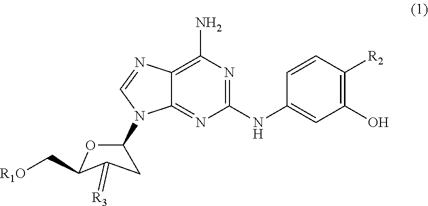
R₁═H or mono- or di- or triphosphate
R₂=butyl- or pentyl- or hexyl- or isohexyl-derivative
R₃═O or NOH 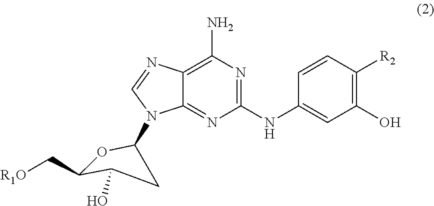
R₁═H or mono- or di- or triphosphate
R₂=butyl- or pentyl- or hexyl- or isohexyl-derivative 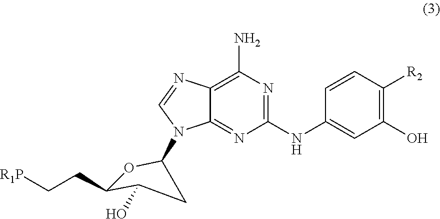
P=phosphonate
R₁═H or mono- or diphosphate
R₂=butyl- or pentyl- or hexy-I and/or isohexyl-derivative. 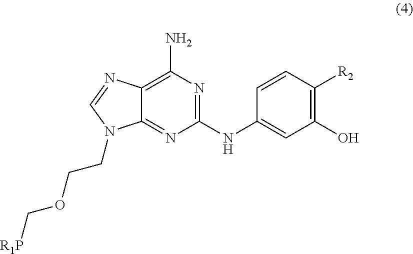
P=phosphonate
R₁═H or mono- or diphosphate
R₂=butyl- or pentyl- or hexy-I and/or isohexyl-derivative, 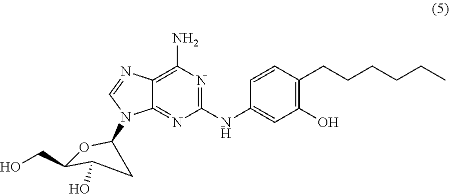
2-(4-Hexyl-3-hydroxyphenylamino)-2′-deoxyadenosine 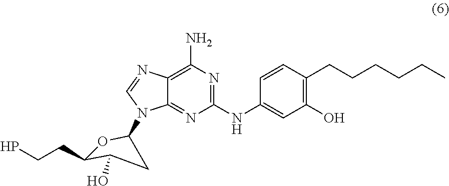
P=phosphonate
2-(4-Hexyl-3-hydroxyphenylamino)-9-[4-hydroxy-5-(2-phosphonoethyl)tetrahydro-2-furyl]adenine 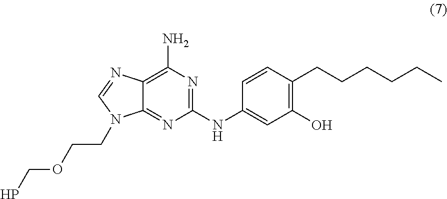
P=phosphonate
2-(4-Hexyl-3-hydroxyphenylamino)-9-[2-(phosphonomethoxy)ethyl]adenine 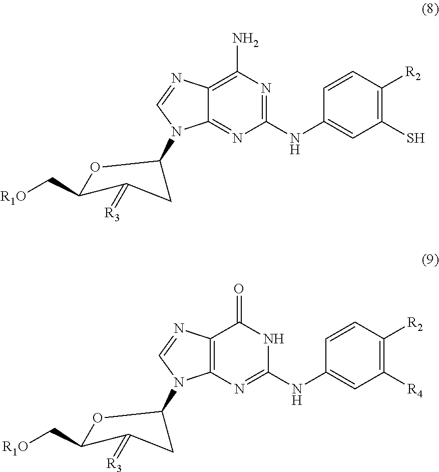
R₁═H or mono- or di- or triphosphate
R₂=butyl- or pentyl- or hexy-I or isohexyl-derivative
R₃═O or NOH
R₄═OH or SH, 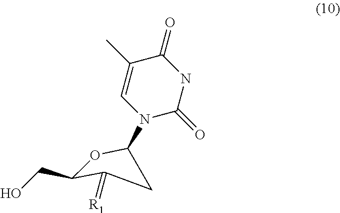
R₁═O or NOH
3′-deoxy-3′-oxothymidine or
3′-deoxy-3′-hydroxyliminothymidine, 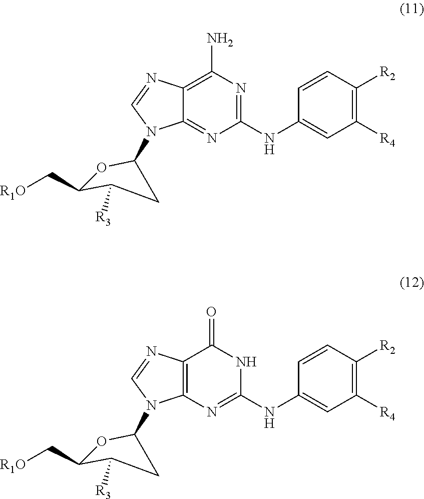
R₁═H or mono- or di- or triphosphate
R₂=butyl- or pentyl- or hexy-I or isohexyl-derivative
R₃═OH or H
R₄═OH or SH, 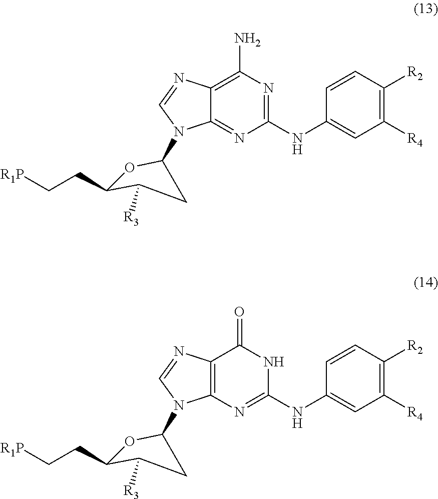
P=phosphonate
R₁═H or mono- or diphosphate
R₂=butyl- or pentyl- or hexy-I or isohexyl-derivative
R₃═OH or H
R₄═OH or SH, 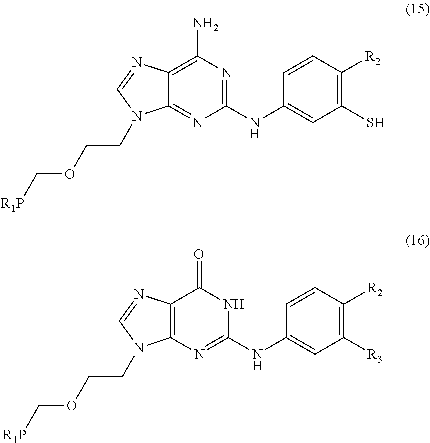
P=phosphonate
R₁═H or mono- or diphosphate
R₂=butyl- or pentyl- or hexy-I or isohexyl-derivative
R₃═OH or SH.

2. The agent according to claim 1, preferably for the treatment of cell modifications, especially in an area of the skin,
wherein the agent is selected from the group comprising compounds according to the general formulas 1 to 4: 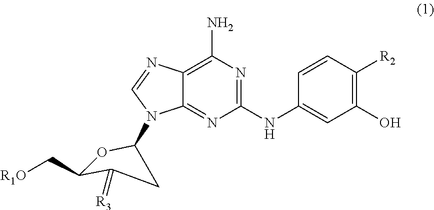
R₁═H or mono- or di- or triphosphate
R₂=butyl- or pentyl- or hexy-I or isohexyl-derivative
R₃═O or NOH 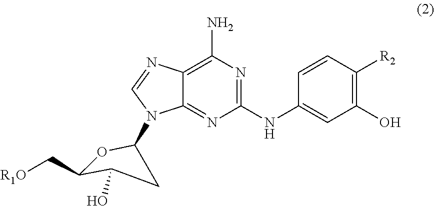
R₁═H or mono- or di- or triphosphate
R₂=butyl- or pentyl- or hexy-I or isohexyl-derivative 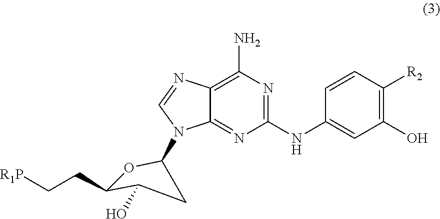
P=phosphonate
R₁═H or mono- or diphosphate
R₂=butyl- or pentyl- or hexy-I and/or isohexyl-derivative. 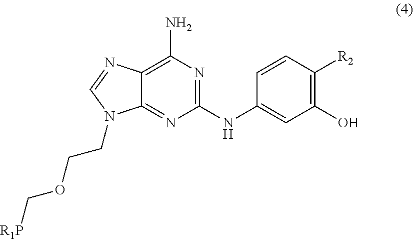
P=phosphonate
R₁═H or mono- or diphosphate
R₂=butyl- or pentyl- or hexy-I and/or isohexyl-derivative.

3. The agent according to claim 1, of general formula 5: 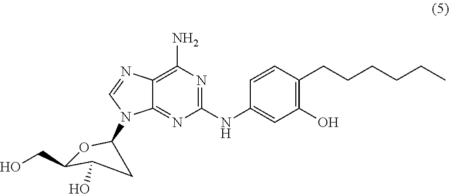
2-(4-Hexyl-3-hydroxyphenylamino)-2′-deoxyadenosine.

4. The agent according to claim 1, of general formula 6: 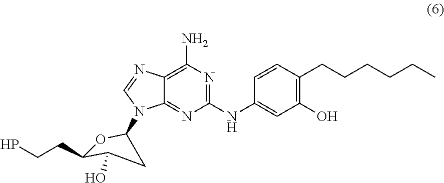
P=phosphonate
2-(4-Hexyl-3-hydroxyphenylamino)-9-[4-hydroxy-5-(2-phosphonoethyl)tetrahydro-2-furyl]adenine

5. The agent according to claim 1, having general formula 7: 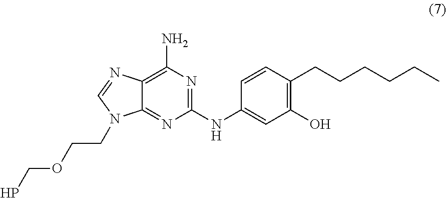
P=phosphonate
2-(4-Hexyl-3-hydroxyphenylamino)-9-[2-(phosphonomethoxy)ethyl]adenine.

6. The agent according to claim 1, wherein the agent is functionally analogous to a compound in accordance with general formulas 1 to 7 and is selected from the group comprising 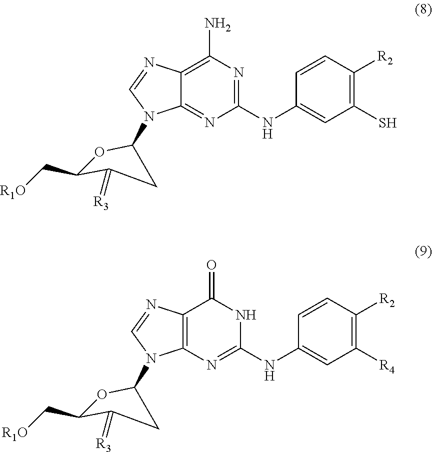
R₁═H or mono- or di- or triphosphate
R₂=butyl- or pentyl- or hexy-I or isohexyl-derivative
R₃═O or NOH
R₄═OH or SH, 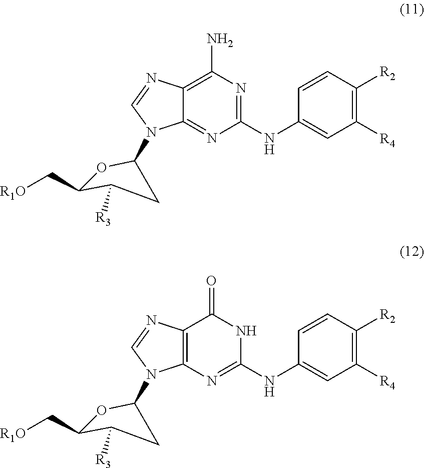
R₁═H or mono- or di- or triphosphate
R₂=butyl- or pentyl- or hexy-I or isohexyl-derivative
R₃═OH or H
R₄═OH or SH, 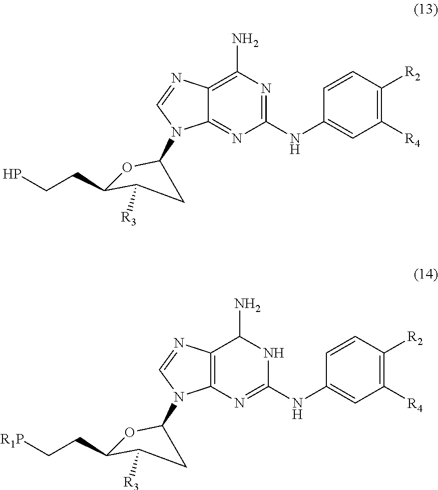
P=phosphonate
R₁═H or mono- or diphosphate
R₂=butyl- or pentyl- or hexy-I or isohexyl-derivative
R₃═OH or H
R₄═OH or SH, 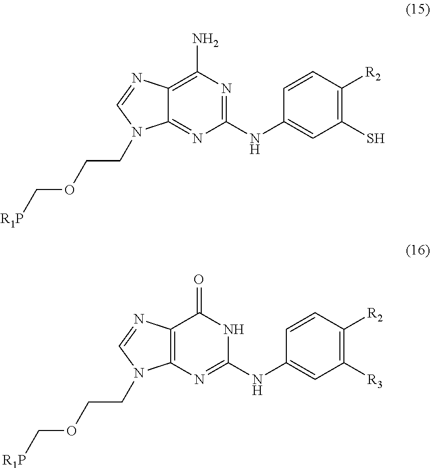
P=phosphonate
R₁═H or mono- or diphosphate
R₂=butyl- or pentyl- or hexy-I or isohexyl-derivative
R₃═OH or SH.

7. An agent of general formula 1 to 9 and 11 to 16 for use as a drug.

8. A pharmaceutical agent comprising the agent according to claim 1 and a pharmaceutically tolerable carrier for treatment of tumors.

9. The pharmaceutical agent according to claim 8,
wherein
the pharmaceutically tolerable carrier is selected from the group comprising fillers, diluents, binders, humectants, dissolution retarders, disintegrants, absorption enhancers, wetting agents, absorbents, lubricants and/or carrier lipids, especially solid lipid nanoparticles, nano-structured lipid carriers, liposomes or polymer particles, preferably dendrimers.

10. A kit comprising the pharmaceutical agent according to claim 8, together with an application system as well as information for combining the contents of the kit.

11. A method of treatment of pathologic cell growth and/or cell growth disorders and/or for modification of a polymerase comprising
administering to a subject in need thereof agents in accordance with general formulas (1) to (16) 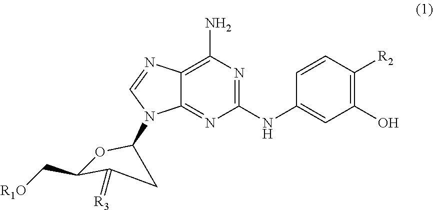
R₁═H or mono- or di- or triphosphate
R₂=butyl- or pentyl- or hexy-I or isohexyl-derivative
R₃═O or NOH 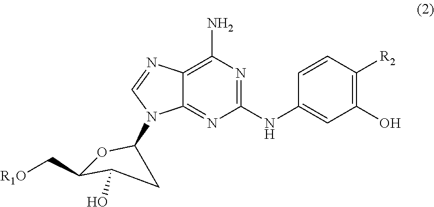
R₁═H or mono- or di- or triphosphate
R₂=butyl- or pentyl- or hexy-I or isohexyl-derivative 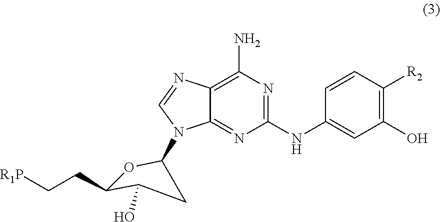
P=phosphonate
R₁═H or mono- or diphosphate
R₂=butyl- or pentyl- or hexy-I and/or isohexyl-derivative. 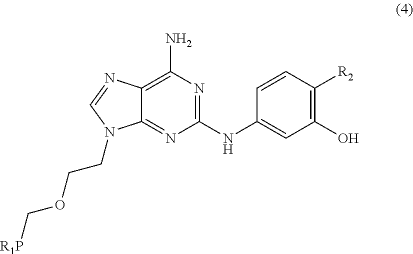
P=phosphonate
R₁═H or mono- or diphosphate
R₂=butyl- or pentyl- or hexy-I and/or isohexyl-derivative, 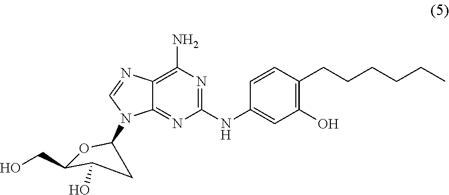
2-(4-Hexyl-3-hydroxyphenylamino)-2′-deoxyadenosine 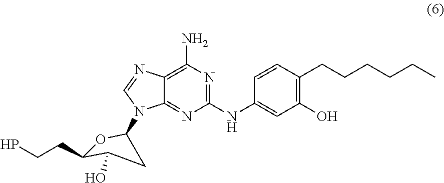
P=phosphonate
2-(4-Hexyl-3-hydroxyphenylamino)-9-[4-hydroxy-5-(2-phosphonoethyl)tetrahydro-2-furyl]adenine 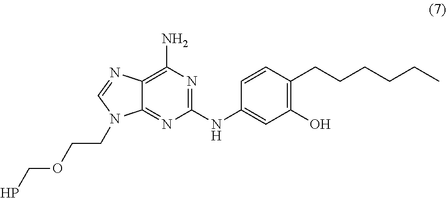
P=phosphonate
2-(4-Hexyl-3-hydroxyphenylamino)-9-[2-(phosphonomethoxy)ethyl]adenine 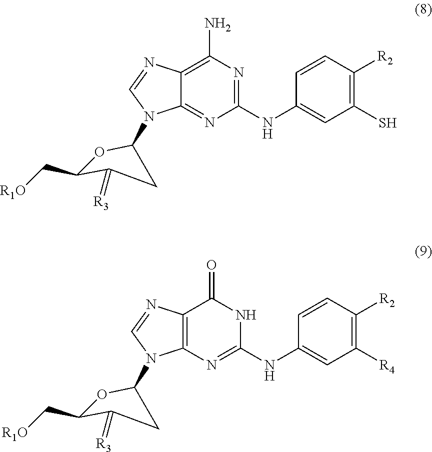
R₁═H or mono- or di- or triphosphate
R₂=butyl- or pentyl- or hexy-I or isohexyl-derivative
R₃═O or NOH
R₄═OH or SH, 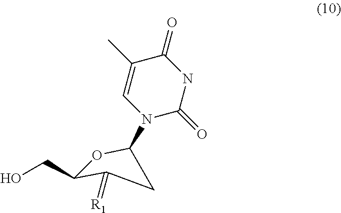
R₁═O or NOH
3′-deoxy-3′-oxothymidine or
3′-deoxy-3′-hydroxyliminothymidine, 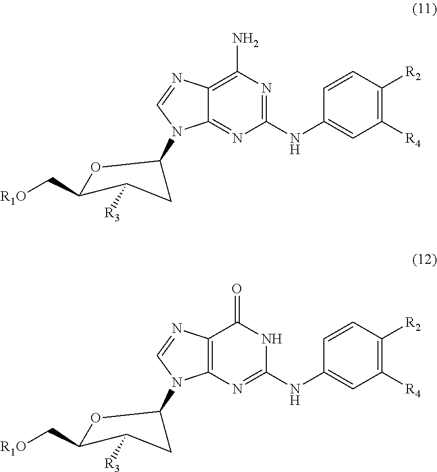
R₁═H or mono- or di- or triphosphate
R₂=butyl- or pentyl- or hexy-I or isohexyl-derivative
R₃═OH or H
R₄═OH or SH, 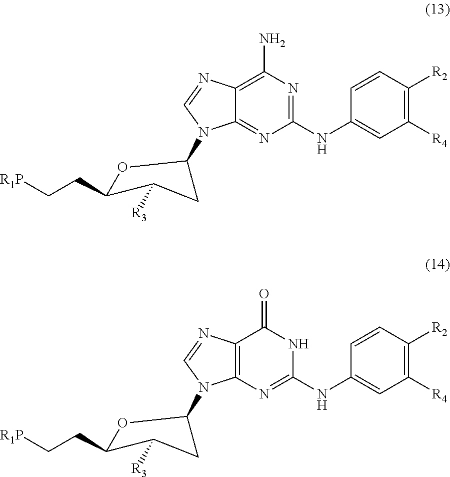
P=phosphonate
R₁═H or mono- or diphosphate
R₂=butyl- or pentyl- or hexy-I or isohexyl-derivative
R₃═OH or H
R₄═OH or SH, 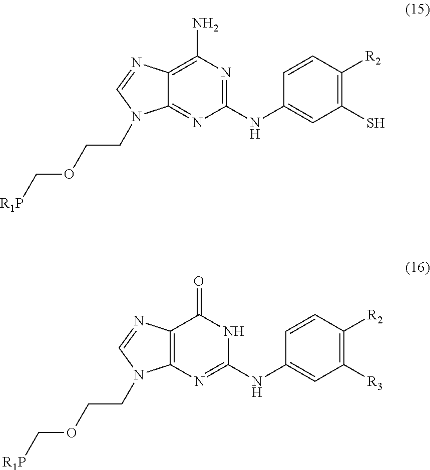
P=phosphonate
R₁═H or mono- or diphosphate
R₂=butyl- or pentyl- or hexy-I or isohexyl-derivative
R₃═OH or SH.
in a treatment of pathologic cell growth and/or cell growth disorders and/or for the modification of a polymerase effective amount.

12. A method according to claim 11,
wherein
the polymerase is a DNA polymerase alpha.

13. The method according to claim 11,
wherein
the cell growth disorder represents cell proliferation.

14. The method according to claim 11,
wherein
the cell proliferation is selected from the group comprising skin cancer of epithelial origin, preferably actinic keratoses, a basalioma or spinalioma, skin cancer of pigment cells, skin cancer of immune cells, fibrosarcomas, perspiratory gland carcinomas, sebaceous gland carcinomas, angiosarcomas, myosarcomas and/or Merkel cell carcinomas.

15. The method according to claim 14,
wherein
the basalioma is a nodular, solid basalioma, a superficial basalioma, a pigmented basalioma, a sclerotizing basalioma, an exulcerating basalioma and/or a destructive basalioma.

16. A method according to claim 11,
wherein
the cell growth disorder is a keratosis.

17. A method according to claim 16,
wherein
the keratosis is seborrhoeic keratosis, actinic keratosis, senile speckles, pigmentation marks and/or lentigo solaris.

18. The method according to claim 11,
wherein
the cell growth disorder is a carcinoma, a sarcoma, a neuroendocrine tumor, a hemooncologic tumor, a dysontogenetic tumor and/or a mixed tumor.

19. The method according to claim 11,
wherein
the agent is provided in the form of a solution, emulsion, suspension, ointment, balm, oil, gel, foam, eye balm, eye gel, suppository, spray, pad, stick or crayon, in liquid form, in the form of artificial tears, thermoreversible gel (to be used in liquid form) and/or in the form of a cream.

20. The method according to claim 11,
wherein the agent is a
pharmaceutical agent comprising a pharmaceutically tolerable carrier in form of a gel, poudrage, powder, tablet, sustained-release tablet, premix, emulsion, brew-up formulation, drops, concentrate, granulate, syrup, pellet, bolus, capsule, aerosol, spray and/or inhalant.

21. The method according to claim 11, wherein the agent has the general formula 7 and is provided as an
ointment is selected from the group comprising unguentum leniens (cooling ointment), unguentum emulsificans (hydrophilic ointment), unguentum emulsificans aquosum (water-containing hydrophilic ointment), unguentum cetomacrogolis, unguentum cetylicum cum aqua (cetyl ointment), unguentum alcoholum lanae or unguentum adeps lanae (wool wax alcohol ointment, eucerine), unguentum molle (soft ointment) and/or unguentum zinci (zinc ointment).

22. A method according to claim 11,
wherein the agent is
a pharmaceutical agent comprising a pharmaceutically tolerable carrier and is present in a preparation at a concentration of from 0.1 to 99.5, preferably from 0.5 to 95.0 and more preferably from 20.0 to 80.0 wt. %.

23. The method according to claim 11,
wherein the agent is
a pharmaceutical agent comprising a pharmaceutically tolerable carrier and is administered in total amounts of from 0.05 to 500 mg per kg, preferably from 5 to 100 mg per kg body weight per 24 hours.

24. A method according to claim 23,
wherein
the pharmaceutical agent is administered orally, subcutaneously, intravenously, intramuscularly, intraperitoneally and/or topically.

25. The method according to claim 11, wherein the agent is administered in an amount effective for a secondary prophylaxis of tumors.

26. The method of the compounds in accordance with formulas (1) to (8) as lead structure for the development of polymerase inhibitors.
